SEQUENCE LISTING (5 sequences)

SEQ ID NO: 1 - DNA, 2049 bp, Magnaportha grisea
atgcttcgtactgttggccgcaaagccctgaggggctcatccaagggatgttcacgaaccatctcgactctcaagcccgccacggcaactattgccaagcccggcagcaggaccctttcgacgccagcgacggcaacagcaacagcacctcgaactaagcccagcgccagcttcaatgctcgccgcgatccccagcctctggtcaaccctcgctcaggtgaggcagacgaatcattcattggcaagacgggaggagagattttccacgagatgatgctgaggcaaaacgtcaagcacatcttcggttaccctggcggtgctatccttcccgtgttcgacgcgatctacaactcgaagcacatcgactttgttctgcccaagcatgagcaaggcgccggccacatggcagagggctatgctcgcgcttcaggcaaacccggcgttgttctcgtcacctccggccccggtgccacaaatgtcatcactcccatggccgacgctcttgccgacggtacacctctggttgtattctcaggacaggttgttacctctgctattggaagcgacgccttccaggaggccgacgtcataggcatctcccggtcttgcaccaagtggaacgtcatggttaagagcgttgacgagctcccgaggagaattaacgaggcctttgagattgccaccagtgggcgacctgggcctgtcttggtcgatctgcccaaggacgtcacggctagtgtgctgaggagggctatccccaccgagacctcgattccctctattagcgctgcagcacgggctgtccaagaggcaggccgaaagcagcttgagcactccatcaaacgcgtagccgatctcgtcaacattgccaagaagcccgtcatatatgccggccaaggtgtcattttgtcggaaggcggcgttgaacttctcaaggcgcttgccgacaaggcctcgattcctgtcaccaccactctgcatggtctgggagcctttgacgagctcgacgagaaggcactgcacatgcttggtatgcacggttcggcttatgccaacatgtccatgcaagaggccgatttgatcattgcccttggtggccgcttcgatgaccgtgtcactggcagcatccccaaatttgctcctgccgccaagcttgctgctgctgaaggccgcggaggtattgtccacttcgagattatgcccaagaacatcaacaaggtcgtccaagcaacagaggccattgagggcgacgttgcttcgaacttgaagctgttgctccccaagattgaacaacgatccatgaccgatcgcaaggagtggttcgaccagatcaaggagtggaaggagaagtggcctctgtcacattatgagagggccgagcgtagtggtctcatcaagcctcagactctgatcgaggagctgagcaacctgactgctgaccgcaaggacatgacctacatcacaaccggtgttggccagcaccaaatgtggacagcacaacatttcaggtggaggcacccacggtccatgatcacctctggcggtttgggtaccatgggatatggtctgccggcagcgattggcgccaaggttgctaggccagatgctttggtcattgacatcgatggcgatgcatcgttcaacatgactctgacagagctttcgacggcggcacagttcaacattggcgtcaaggtcattgtcttgaacaacgaggagcagggaatggtgacccaatggcagaacttgttctacgaggaccgctactcacatacacaccagcgcaacccagacttcatgaagctcgccgatgcaatggacgttcaacatcgccgtgtttcgaagcctgacgatgtcgttgatgctctgacgtggctgatcaacaccgacggccccgctctgctcgaggtgatgactgataagaaggttcctgttctgcccatggtgcccggaggtaacggcctgcacgagttcatcacgtttgatgccagcaaggataagcaacggagagagctgatgcgcgcgaggacgaacggcctgcacggttaa

SEQ ID NO: 2 - protein, 682 aa, Magnaportha grisea
MLRTVGRKALRGSSKGCSRTISTLKPATATIAKPGSRTLSTPATATATAPRTKPSASFNARRDPQPLVNPRSGEADESFIGKTGGEIFHEMMLRQNVKHIFGYPGGAILPVFDAIYNSKHIDFVLPKHEQGAGHMAEGYARASGKPGVVLVTSGPGATNVITPMADALADGTPLVVFSGQVVTSAIGSDAFQEADVIGISRSCTKWNVMVKSVDELPRRINEAFEIATSGRPGPVLVDLPKDVTASVLRRAIPTETSIPSISAAARAVQEAGRKQLEHSIKRVADLVNIAKKPVIYAGQGVILSEGGVELLKALADKASIPVTTTLHGLGAFDELDEKALHMLGMHGSAYANMSMQEADLIIALGGRFDDRVTGSIPKFAPAAKLAAAEGRGGIVHFEIMPKNINKVVQATEAIEGDVASNLKLLLPKIEQRSMTDRKEWFDQIKEWKEKWPLSHYERAERSGLIKPQTLIEELSNLTADRKDMTYITTGVGQHQMWTAQHFRWRHPRSMITSGGLGTMGYGLPAAIGAKVARPDALVIDIDGDASFNMTLTELSTAAQFNIGVKVIVLNNEEQGMVTQWQNLFYEDRYSHTHQRNPDFMKLADAMDVQHRRVSKPDDVVDALTWLINTDGPALLEVMTDKKVPVLPMVPGGNGLHEFITFDASKDKQRRELMRARTNGLHG

SEQ ID NO: 3 - DNA, 951 bp, Magnaportha grisea
atggcttctcgcagcctgctctcgtcgacatggcgcgccgccaccgccgcggcactccgacccacggccgccgtcagacacagctcgagtagctcgacgtcggccatcgcctacaaggcccttcgccgtcgccaagcgccgctcccgaccagcgactctccgcccgcttggtcatctgcccaggctgccgtctctaacattctctacgagaccccgacgccgtccacggctcctccaaagaggcacatcctcaactgcctggttcagaacgagcccggtgtcctgtcccgcgtttccggaattctcgccgcccgtgggttcaacatcgactcactcgtcgtctgcagcaccgaggtcgcggacctgtcgcgcatgacgatcgtgctgacggggcaggacggcgtcgtcgagcaggccaggcggcagctggaggacctcgtccccgtctgggccgtgctggactacaccgactcaccgctagtccagagggagctcctccttgccaaggtcaacatcttggggcaggagtactttgaggagctgctggaccaccatcgcgagatcaccagcgccgacgctgaagtcctggcctccagcgagggagagcagaccttggagcaagtggctgctgacttccacccgagcaggttggccgctagcgaggctttgcgccacaagcacgagcacctcaagagcatcacgtactttacgcatcagtttggtggaaaggttttggacattagcacaaacagctgcattgttgaactgtctgcaaaaccctcgaggattgactcgttcctcaagttgattggccccttcggtattcttgagtccgcgaggactggtctgatggcactgccgcggtcgcccctgtatgggcctaatgaagaggaagctctcatcaaggaggctgatgacattgttgacgccagccagctgccccctggttaa

SEQ ID NO: 4 - DNA, 3072 bp, Magnaportha grisea
ggtcagaatatggcgcggtcaagatggacagggaagccagaatgtataagtaggtacagacctgcggcagcggcaggatccagagcagcagtagaaggttcctcgctcttctttgcttcgtcatcggcgcgagggagaggactgcgatcgcgtcctggctcagactttcgggaggggggcgatcgggagcgtcggtcgaatctcgaagtccttcgaggttccggctcggtctggtcgaagcgcgaacgcttaggacggggctctgcgtcggccatggtgtttttgaatttgactgttgtctgttgcaatgcggaccctccgctttcgtatcggtaccaattcttgaccaggtctgttaatgaactcgaattcctcttttaccaaaaaggctacaggcgccctggcggacgtctgactttgttgtaaaggatgatgatgtggtttcaagtgatttgctacacacggctacgactgtgcagcgcgtctggaaaaattggaaacttctttggtgagcgagctccgcgggtggggattgcactaagccaaaaatagaaatttattttcggcaggattagttgggtggggattcacgatcttagagctccgaccagtctcaactaaagtcagtcacaaattcctttgttattactccgtataattcaagttcaaagtcacctactatgaaagtacctatggatgagaagtggcccaattgattaggtcaagagttctcaactgccaacaaactgacatttaaatggcatttaactcttgatttgtcttggatcatagccatcaagttaaattatcacctcaatcttgcgcttatccaacacggctgtatttaaactttgactacacctttgacctgcaacgcttgagctgagctgtattagacttcggtctagttctggattttctgctgctcaggacgttggagcgtacaccaaatctcttgaataacatttcaccgcagcaaagtccaatgcacttcgagtggaagtgacccagtgcagctatcaagggtccgatttcatcggaatacgttctcgattcagtcagtatacggacgggtacctgaaattttggtcatgcagcattgtgactagcagaagcatattcgaattcgtcgcaacaaaaatgatatgttgctgaacatgcaaatcatgactagaaacacaatttggcgtaatcgtggttggtagttttcttttgcgtgtgtttaccgtaggtatgcttgtctagaaacggcctggactaggtctagaatctcagagtctagcctgtcaagtctgataccacctcagctccgctatcggcgatccaggcccgacaatcaattaaattcgacccattaatattgactctttctctccagaaacaaaaagaaaagatcacaacgacaccttcgtcaacgttgagtcacgttacacagtctctcaaaggtcaattgatcgcgataaagtgcttacttataaaggagcacagcctaaacacatttttcgcaatccaagaggcatggcttctcgcagcctgctctcgtcgacatggcgcgccgccaccgccgcggcactccgacccacggccgccgtcagacacagctcgagtagctcgacgtcggccatcgcctacaaggcccttcgccgtcgccaagcgccgctcccgaccagcgactctccgcccgcttggtcatctgcccaggctgccgtctctaacattctctacgagaccccgacgccgtccacggctcctccaaagaggcacatcctcaactgcctggttcagaacgagcccggtgtcctgtcccgcgtttccggaattctcgccgcccgtgggttcaacatcgactcactcgtcgtctgcagcaccgaggtcgcggacctgtcgcgcatgacgatcgtgctgacggggcaggacggcgtcgtcgagcaggccaggcggcagctggaggacctcgtccccgtctgggccgtgctggactacaccgactcaccgctagtccagagggagctcctccttgccaaggtcaacatcttggggcaggagtactttgaggagctgctggaccaccatcgcgagatcaccagcgccgacgctgaagtcctggcctccagcgagggagagcagaccttggagcaagtggctgctgacttccacccgagcaggttggccgctagcgaggctttgcgccacaagcacgagcacctcaagagcatcacgtactttacgcatcagtttggtggaaaggttttggacattagcacaaacagctgcattgttgaactgtgagtattgaacacctgttcatttttttttgtttggctcgtgtcaattgtgcaaatatactaacactagttggtaaggtctgcaaaaccctcgaggattgactcgttcctcaagttgattggccccttcggtattcttgagtccgcgaggactggtctgatggcactgccgcggtcgcccctgtatgggcctaatgaagaggaagctctcatcaaggaggctgatgacattgttgacgccagccagctgccccctggttaaagggacttaatcgaatgagaactagaactagagcagcagattgaatgtaatcttttactcatacatgacattcagtcactttgtgcatgtgctgtgggaaagagagaggcagacagcatttgtttgcctctgagcttgttttttcagttgcctcaaccgggatgacaaagcaactaccccagtgtcacacgagcagatcaatggagaggggcgttcctggctctatccagtatcgtattctcaattgccggtaacgaactacttgcccgcccacatacttactttttgaacctgcattagagaagcaatcactatgcagggtaggtgggtgggtgacccggcagagcaaaagtggcacaatgccagatcggcttacgatctggctggagtggggcacctcagctaagggtgccagtatttgggtatctcagcagctagcagggatgcaaaacgatttccagaaacaattaattatcctgcttcgtattcccaaatgataccttgacttggaaccttgctcccttt

SEQ ID NO: 5 - protein, 316 aa, Magnaportha grisea
MASRSLLSSTWRAATAAALRPTAAVRHSSSSSTSAIAYKALRRRQAPLPTSDSPPAWSSAQAAVSNILYETPTPSTAPPKRHILNCLVQNEPGVLSRVSGILAARGFNIDSLVVCSTEVADLSRMTIVLTGQDGVVEQARRQLEDLVPVWAVLDYTDSPLVQRELLLAKVNILGQEYFEELLDHHREITSADAEVLASSEGEQTLEQVAADFHPSRLAASEALRHKHEHLKSITYFTHQFGGKVLDISTNSCIVELSAKPSRIDSFLKLIGPFGILESARTGLMALPRSPLYGPNEEEALIKEADDIVDASQLPPG

(19) United States
(12) Patent Application Publication
(10) Pub. No.: US 2005/0048593 A1
(43) Pub. Date: Mar. 3, 2005

(54) Methods for the identification of inhibitors of acetolactate synthase as antibiotics

(76) Inventors: Todd M. DeZwaan, Apex, NC (US); Sze-Chung C. Lo, Shun Lee Estate (HK); Maria V. Montenegro-Chamorro, Durham, NC (US); Blaise A. Darveaux, Hillsborough, NC (US); Sheryl A. Frank, Durham, NC (US); Ryan W. Heiniger, Holly Springs, NC (US); Sanjoy K. Mahanty, Chapel Hill, NC (US); Huaqin Pan, Apex, NC (US); Amy S. Covington, Raleigh, NC (US); Rex Tarpey, Apex, NC (US); Jeffrey R. Shuster, Chapel Hill, NC (US); Matthew M. Tanzer, Durham, NC (US); Lisbeth Hamer, Durham, NC (US); Kiichi Adachi, Osaka (JP)

Correspondence Address: Icoria, Inc., 108 T.W. ALEXANDER DRIVE, P O BOX 14528, RTP, NC, 27709-4528, US

(21) Appl. No.: 10/877,284

(22) Filed: Jun. 25, 2004

Related U.S. Application Data
(60) Provisional application No. 60/483,340, filed on Jun. 27, 2003.

(51) Int. Cl. C12Q 1/48; C12Q 1/18

(52) U.S. Cl. 435/15; 435/32

(57) ABSTRACT

The present inventors have discovered that ALS catalytic and regulatory subunits are essential for normal fungal pathogenicity. Specifically, the inhibition of either ALS catalytic or regulatory subunit gene expression in fungi severely reduces growth and pathogenicity. Thus, ALS catalytic and regulatory subunits are useful as targets for the identification of antibiotics, preferably antifungals. Accordingly, the present invention provides methods for the identification of compounds that inhibit ALS catalytic or regulatory subunit expression or activity. The methods of the invention are useful for the identification of antibiotics, preferably antifungals.

### RELATED APPLICATIONS

This application claims the benefit of U.S. Provisional Application No. 60/483,340, filed Jun. 27, 2003, which is incorporated in entirety by reference.

### FIELD OF THE INVENTION

The invention relates generally to methods for the identification of antibiotics, preferably antifungals that affect the biosynthesis of branched chain amino acids.

### BACKGROUND OF THE INVENTION

Filamentous fungi are causal agents responsible for many serious pathogenic infections of plants and animals. Since fungi are eukaryotes, and thus more similar to their host organisms than, for example bacteria, the treatment of infections by fungi poses special risks and challenges not encountered with other types of infections. One such fungus is Magnaporthe grisea, the fungus that causes rice blast disease, a significant threat to food supplies worldwide. Other examples of plant pathogens of economic importance include the pathogens in the genera Agaricus, Alternaria, Anisogramma, Anthracoidea, Antrodia, Apiognomonia, Apiosporina, Armillaria, Ascochyta, Aspergillus, Bipolaris, Bjerkandera, Botryosphaeria, Botrytis, Ceratobasidium, Ceratocystis, Cercospora, Cercosporidium, Cerotelium, Cerrena, Chondrostereum, Chryphonectria, Chrysomyxa, Cladosporium, Claviceps, Cochliobolus, Coleosporium, Colletotrichium, Colletotrichum, Corticium, Corynespora, Cronartium, Cryphonectria, Cryptosphaeria, Cyathus, Cymadothea, Cytospora, Daedaleopsis, Diaporthe, Didymella, Diplocarpon, Diplodia, Discohainesia, Discula, Dothistroma, Drechslera, Echinodontium, Elsinoe, Endocronartium, Endothia, Entyloma, Epichloe, Erysiphe, Exobasidium, Exserohilum, Fomes, Fomitopsis, Fusarium, Gaeumannomyces, Ganoderma, Gibberella, Gloeocercospora, Gloeophyllum, Gloeoporus, Glomerella, Gnomoniella, Guignardia, Gymnosporangium, Helminthosporium, Herpotrichia, Heterobasidion, Hirschioporus, Hypodermella, Inonotus, Irpex, Kabatiella, Kabatina, Laetiporus, Laetisaria, Lasiodiplodia, Laxitextum, Leptographium, Leptosphaeria, Leptosphaerulina, Leucytospora, Linospora, Lophodermella, Lophodermium, Macrophomina, Magnaporthe, Marssonina, Melampsora, Melampsorella, Meria, Microdochium, Microsphaera, Monilinia, Monochaetia, Morchella, Mycosphaerella, Myrothecium, Nectria, Nigrospora, Ophiosphaerella, Ophiostoma, Penicillium, Perenniporia, Peridermium, Pestalotia, Phaeocryptopus, Phaeolus, Phakopsora, Phellinus, Phialophora, Phoma, Phomopsis, Phragmidium, Phyllachora, Phyllactinia, Phyllosticta, Phymatotrichopsis, Pleospora, Podosphaera, Pseudopeziza, Pseudoseptoria, Puccinia, Pucciniastrum, Pyricularia, Rhabdocline, Rhizoctonia, Rhizopus, Rhizosphaera, Rhynchosporium, Rhytisma, Schizophyllum, Schizopora, Scirrhia, Sclerotinia, Sclerotium, Scytinostroma, Septoria, Setosphaera, Sirococcus, Spaerotheca, Sphaeropsis, Sphaerotheca, Sporisorium, Stagonospora, Stemphylium, Stenocarpella, Stereum, Taphrina, Thielaviopsis, Tilletia, Trametes, Tranzschelia, Trichoderma, Tubakia, Typhula, Uncinula, Urocystis, Uromyces, Ustilago, Valsa, Venturia, Verticillium, Xylaria, and others. Related organisms are classified in the oomycetes classification and include the genera Albugo, Aphanomyces, Bremia, Peronospora, Phytophthora, Plasmodiophora, Plasmopara, Pseudoperonospora, Pythium, Sclerophthora, and others. Oomycetes are also significant plant pathogens and are sometimes classified along with the true fungi.

Human diseases that are caused by filamentous fungi include life-threatening lung and disseminated diseases, often a result of infections by Aspergillus fumigatus. Other fungal diseases in animals are caused by fungi in the genera Fusarium, Blastomyces, Microsporum, Trichophyton, Epidermophyton, Candida, Histoplamsa, Pneumocystis, Cryptococcus, other Aspergilli, and others. Control of fungal diseases in plants and animals is usually mediated by chemicals that inhibit growth, proliferation, and/or pathogenicity of fungal organisms. To date, there are less than twenty known modes-of-action for plant protection fingicides and human antifungal compounds.

A pathogenic organism has been defined as an organism that causes, or is capable of causing disease. Pathogenic organisms propagate on or in tissues and may obtain nutrients and other essential materials from their hosts. A substantial amount of work concerning filamentous fungal pathogens has been performed with the human pathogen, Aspergillus fumigatus. Shibuya et al., 27 Microb. Pathog. 123 (1999) (PubMed Identifier (PMID): 10455003) have shown that the deletion of either of two suspected pathogenicity related genes encoding an alkaline protease or a hydrophobin (rodlet), respectively, did not reduce mortality of mice infected with these mutant strains. Smith et al., 62 Infect. Immun. 5247 (1994) (PMID: 7960101) showed similar results with alkaline protease and the ribotoxin restrictocin; Aspergillus fumigatus strains mutated for either of these genes were fully pathogenic to mice. Reichard et al., 35 J. Med. Vet. Mycol. 189 (1997) (PMID: 9229335) showed that deletion of the suspected pathogenicity gene encoding aspergillopepsin (PEP) in Aspergillus fumigatus had no effect on mortality in a guinea pig model system, whereas Aufauvre-Brown et al., 21 Fungal. Genet. Biol. 141 (1997) (PMID: 9073488) showed no effects of a chitin synthase mutation on pathogenicity.

However, not all experiments produced negative results. Ergosterol is an important membrane component found in fungal organisms. Pathogenic fungi lacking key enzymes in the ergosterol biochemical pathway might be expected to be non-pathogenic since neither the plant nor animal hosts contain this particular sterol. Many antifungal compounds that affect the ergosterol biochemical pathway have been previously described. (U.S. Pat. Nos. 4,920,109; 4,920,111; 4,920,112; 4,920,113; and 4,921,844; Hewitt, H. G. Fungicides in Crop Protection Cambridge, University Press (1998)). D'Enfert et al., 64 Infect. Immun. 4401 (1996) (PMID: 8926121)) showed that an Aspergillus fumigatus strain mutated in an orotidine 5′-phosphate decarboxylase gene was entirely non-pathogenic in mice, and Brown et al. (Brown et al., 36 Mol. Microbiol. 1371 (2000) (PMID: 10931287)) observed a non-pathogenic result when genes involved in the synthesis of para-aminobenzoic acid were mutated. Some specific target genes have been described as having utility for the screening of inhibitors of plant pathogenic fungi. U.S. Pat. No. 6,074,830 to Bacot et al., describe the use of 3,4-dihydroxy-2-butanone 4-phosphate synthase, and U.S. Pat. No. 5,976,848 to Davis et al. describes the use of dihydroorotate dehydrogenase for potential screening purposes.

There are also a number of papers that report less clear results, showing neither full pathogenicity nor non-pathogenicity of mutants. For example, Hensel et al. (Hensel, M. et al., 258 Mol. Gen. Genet. 553 (1998) (PMID: 9669338)) showed only moderate effects of the deletion of the area transcriptional activator on the pathogenicity of Aspergillus fumigatus. Therefore, it is not currently possible to determine which specific growth materials may be readily obtained by a pathogen from its host, and which materials may not.

The present invention discloses polypeptides in the branched chain amino acid biosynthetic pathway for the identification of antifungal, biocide, and biostatic materials.

### SUMMARY OF THE INVENTION

The present inventors have discovered that in vivo disruption of ILV2 or ILV6 genes encoding acetolactate synthase catalytic and regulatory subunits in Magnaporthe grisea, respectively, greatly reduces the growth and pathogenicity of the fungus. Thus, the present inventors have discovered that acetolactate synthase enzyme (ALS) and each of the ALS catalytic and regulatory subunits alone are useful as targets for the identification of antibiotics, preferably fungicides. Accordingly, the present invention provides methods for the identification of compounds that inhibit acetolactate synthase expression or activity. Methods of the present invention are useful for the identification of antibiotics, preferably fungicides.

### BRIEF DESCRIPTION OF THE FIGURES

FIG. 1. Diagram of the reversible reaction catalyzed by acetolactate synthase (ALS). The ALS enzyme contains a regulatory subunit and a larger catalytic subunit and catalyzes the interconversion of two pyruvate molecules with 2-acetolactate and CO₂. ALS activity is part of the branched chain amino acid biosynthesis pathway. The ALS regulatory subunit functions to stimulate ALS enzymatic activity and is believed to have a role in feedback regulation and/or enzymatic stability (Pang S. S. and Duggleby R. G., 38 Biochemistry 5222-31(1999)).

FIG. 2. Digital image showing the effect of ILV2 gene disruption on Magnaporthe grisea pathogenicity using whole plant infection assays. Rice variety CO39 was inoculated with wild-type strain Guyll, transposon insertion strains, K1-13 and K1-19. Leaf segments were imaged at seven days post-inoculation. Mutants K1-13 and K1-19 showed reduced pathogenicity (i.e. smaller, non-viable lesions) compared to the larger viable lesions of wild type strain Guy11.

FIG. 3. Digital image showing the effect of ILV6 gene disruption on Magnaporthe grisea pathogenicity using whole plant infection assays. Rice variety CO39 was inoculated with wild-type strain Guy11, transposon insertion strains, K1-6 and K1-11. Leaf segments were imaged at seven days post-inoculation. Mutants K1-6 and K1-11 showed reduced pathogenicity (i.e. smaller, non-viable lesions) compared to the larger viable lesions of wild type strain Guy11.

FIG. 4. Image displaying the results of a growth/nutritional requirement analysis of mutant ILV6 Magnaporthe grisea strains, K1-6 and K1-11. Plate (A) displays fungal growth on minimal media and plate (B) displays fungal growth on minimial media supplemented with 4 mM each of isoleucine, leucine, and valine. Growth of all strains was normal on the supplemented media, while growth of strains K1-11 and K1-6 was inhibited as compared to wild type (Guy11) on minimal media alone.

### DETAILED DESCRIPTION OF THE INVENTION

Unless otherwise indicated, the following terms are intended to have the following meanings in interpreting the present invention.

As used herein, the term “acetolactate synthase (ALS) catalytic subunit,” refers to a catalytic subunit of an ALS enzyme that catalyzes the reversible interconversion of two pyruvate molecules with 2-acetolactate and CO₂. Although the protein and/or the name of the gene that encodes the protein may differ between species, the terms “ALS catalytic subunit,” “ILV2 gene product,” and “acetohydroxy acid synthase (AHAS) catalytic subunit” are intended to encompass any polypeptide that catalyzes the reversible interconversion of two pyruvate molecules with 2-acetolactate and CO₂. For example, the phrase “ALS catalytic subunit gene” includes the ILV2 gene from M. grisea as well as genes from other organisms that encode a polypeptide that catalyzes the reversible interconversion of two pyruvate molecules with 2-acetolactate and CO₂, regardless of whether or not the genes from other organisms are referred to as “ILV2”.

As used herein, the terms “acetolactate synthase (ALS) enzyme” and “acetolactate (ALS) regulatory/catalytic subunit complex” and “acetolactate synthase (ALS) holo enzyme” are used interchangeably and refer to an enzyme comprising a regulatory and a catalytic subunit that catalyzes the reversible interconversion of two pyruvate molecules with 2-acetolactate and CO₂.

As used herein, the terms “acetolactate synthase (ALS) regulatory subunit” and “acetolactate synthase (ALS) small subunit” are interchangeable and refer to a regulatory subunit of an enzyme that catalyzes the reversible interconversion of two pyruvate molecules with 2-acetolactate and CO₂. The phrase “regulatory subunit” means a polypeptide capable of increasing enzymatic activity of an ALS catalytic subunit in the absence of amino acids. Although the protein and/or the name of the gene that encodes the protein may differ between species, the terms “ALS regulatory subunit”, “acetohydroxy acid synthase (AHAS) regulatory subunit” and “ILV6 gene product” are intended to encompass any polypeptide that is a regulatory subunit of an enzyme that catalyzes the reversible interconversion of two pyruvate molecules with 2-acetolactate and CO₂. For example, the phrase “ALS regulatory subunit gene” includes the ILV6 gene from M. grisea as well as genes from other organisms that encode a polypeptide that is a regulatory subunit of an enzyme that catalyzes the reversible interconversion of two pyruvate molecules with 2-acetolactate and CO₂, regardless of whether or not the genes from other organisms are referred to as “ILV6”.

The term “antibiotic” refers to any substance or compound that when contacted with a living cell, organism, virus, or other entity capable of replication, results in a reduction of growth, viability, or pathogenicity of that entity.

The term “antipathogenic,” as used herein, refers to a mutant form of a gene that inactivates a pathogenic activity of an organism on its host organism or substantially reduces the level of pathogenic activity, wherein “substantially” means a reduction at least as great as the standard deviation for a measurement, preferably a reduction to 50% activity, more preferably a reduction of at least one magnitude, i.e. to 10% activity. The pathogenic activity affected may be an aspect of pathogenic activity governed by the normal form of the gene, or the pathway the normal form of the gene functions on, or the pathogenic activity of the organism in general. “Antipathogenic” may also refer to a cell, cells, tissue, or organism that contains the mutant form of a gene; a phenotype associated with the mutant form of a gene, and/or associated with a cell, cells, tissue, or organism that contain the mutant form of a gene.

The term “binding” refers to a non-covalent or a covalent interaction, preferably non-covalent, that holds two molecules together. For example, two such molecules could be an enzyme and an inhibitor of that enzyme. Non-covalent interactions include hydrogen bonding, ionic interactions among charged groups, van der Waals interactions, and hydrophobic interactions among nonpolar groups. One or more of these interactions can mediate the binding of two molecules to each other.

The term “biochemical pathway” or “pathway” refers to a connected series of biochemical reactions normally occurring in a cell. Typically, the steps in such a biochemical pathway act in a coordinated fashion to produce a specific product or products or to produce some other particular biochemical action. Such a biochemical pathway requires the expression product of a gene if the absence of that expression product either directly or indirectly prevents the completion of one or more steps in that pathway, thereby preventing or significantly reducing the production of one or more normal products or effects of that pathway. Thus, an agent specifically inhibits such a biochemical pathway requiring the expression product of a particular gene if the presence of the agent stops or substantially reduces the completion of the series of steps in that pathway. Such an agent may, but does not necessarily, act directly on the expression product of that particular gene.

As used herein, the term “conditional lethal” refers to a mutation permitting growth and/or survival only under special growth or environmental conditions.

As used herein, the term “cosmid” refers to a hybrid vector used in gene cloning that includes a cos site (from the lambda bacteriophage). In some cases, the cosmids of the invention comprise drug resistance marker genes and other plasmid genes. Cosmids are especially suitable for cloning large genes or multigene fragments. “Fungi” (singular: fungus) refers to whole fungi, fungal organs and tissues (e.g., asci, hyphae, pseudohyphae, rhizoid, sclerotia, sterigmata, spores, sporodochia, sporangia, synnemata, conidia, ascostroma, cleistothecia, mycelia, perithecia, basidia and the like), spores, fungal cells and the progeny thereof. Fungi are a group of organisms (about 50,000 known species), including, but not limited to, mushrooms, mildews, moulds, yeasts, etc., comprising the kingdom Fungi. Fungi exist as single cells or make up a multicellular body called a mycelium, which consists of filaments known as hyphae. Most fungal cells are multinucleate and have cell walls composed chiefly of chitin. Fungi exist primarily in damp situations on land and, lacking the ability to manufacture their own food by photosynthesis due to an absence of chlorophyll, are either parasites on other organisms or saprotrophs feeding on dead organic matter. Principal criteria used in classification are the nature of the spores produced and the presence or absence of cross walls within the hyphae. Fungi are distributed worldwide in terrestrial, freshwater, and marine habitats. Some fungi live in the soil. Many pathogenic fungi cause disease in animals and man or in plants, while some saprotrophs are destructive to timber, textiles, and other materials. Some fungi form associations with other organisms, most notably with algae to form lichens.

As used herein, the term “fungicide,” “antifungal,” or “antimycotic” refers to an antibiotic substance or compound that kills or suppresses the growth, viability, or pathogenicity of at least one fungus, fungal cell, fungal tissue or spore.

In the context of this disclosure, “gene” should be understood to refer to a unit of heredity. Each gene is composed of a linear chain of deoxyribonucleotides that can be referred to by the sequence of nucleotides forming the chain. Thus, “sequence” is used to indicate both the ordered listing of the nucleotides that form the chain, and the chain having that sequence of nucleotides. “Sequence” is used in the similar way in referring to RNA chains, linear chains made of ribonucleotides. The gene may include regulatory and control sequences, sequences that can be transcribed into an RNA molecule, and may contain sequences with unknown function. The majority of the RNA transcription products are messenger RNAs (mRNAs), which include sequences that are translated into polypeptides and may include sequences that are not translated. It should be recognized that small differences in nucleotide sequence for the same gene can exist between different fungal strains, or even within a particular fungal strain, without altering the identity of the gene.

As used in this disclosure, the terms “growth” or “cell growth” of an organism refer to an increase in mass, density, or number of cells of the organism. Common methods for the measurement of growth include the determination of the optical density of a cell suspension, the counting of the number of cells in a fixed volume, the counting of the number of cells by measurement of cell division, the measurement of cellular mass or cellular volume, and the like.

As used in this disclosure, the term “growth conditional phenotype” indicates that a fungal strain having such a phenotype exhibits a significantly greater difference in growth rates in response to a change in one or more of the culture parameters than an otherwise similar strain not having a growth conditional phenotype. Typically, a growth conditional phenotype is described with respect to a single growth culture parameter, such as temperature. Thus, a temperature (or heat-sensitive) mutant (i.e., a fungal strain having a heat-sensitive phenotype) exhibits significantly different growth, and preferably no growth, under non-permissive temperature conditions as compared to growth under permissive conditions. In addition, such mutants preferably also show intermediate growth rates at intermediate, or semi-permissive, temperatures. Similar responses also result from the appropriate growth changes for other types of growth conditional phenotypes.

As used herein, the term “heterologous ALS catalytic subunit” means either a nucleic acid encoding a polypeptide or a polypeptide, wherein the polypeptide has at least 40%, 45%, 50%, 55%, 60%, 65%, 70%, 75%, 80%, 85%, 90%, 91%, 92%, 93%, 94%, 95%, 96%, 97%, 98%, or 99% sequence identity or each integer unit of sequence identity from 40-99% in ascending order to M. grisea ALS catalytic subunit protein (SEQ ID NO:2) and at least 10%, 25%, 50%, 75%, 80%, 90%, 95%, or 99% activity or each integer unit of activity from 10-100% in ascending order of the activity of the M. grisea protein (SEQ ID NO:2). Examples of heterologous ALS catalytic subunits include, but are not limited to, ALS catalytic subunit from Neurospora crassa and ALS catalytic subunit from Saccharomyces cerevisiae.

As used herein, the term “heterologous ALS regulatory subunit” means either a nucleic acid encoding a polypeptide or a polypeptide, wherein the polypeptide has at least 40%, 45%, 50%, 55%, 60%, 65%, 70%, 75%, 80%, 85%, 90%, 91%, 92%, 93%, 94%, 95%, 96%, 97%, 98%, or 99% sequence identity or each integer unit of sequence identity from 40-99% in ascending order to M. grisea ALS regulatory subunit protein (SEQ ID NO:5) and at least 10%, 25%, 50%, 75%, 80%, 90%, 95%, or 99% activity or each integer unit of activity from 10-100% in ascending order of the activity of the M. grisea protein (SEQ ID NO:5). A polypeptide having at least 10% of the activity of M. grisea ALS regulatory subunit protein (SEQ ID NO:5) is a polypeptide capable of increasing enzymatic activity of an ALS catalytic subunit in the absence of amino acids and/or reducing enzymatic activity of the ALS catalytic subunit in the presence of valine by at least 10% relative to the polypeptide set forth in SEQ ID NO:5. Examples of heterologous ALS regulatory subunits include, but are not limited to, ALS regulatory subunit from Neurospora crassa and ALS regulatory subunit from Saccharomyces cerevisiae.

As used herein, the term “His-Tag” refers to an encoded polypeptide consisting of multiple consecutive histidine amino acids.

As used herein, the terms “hph,” “hygromycin B phosphotransferase,” and “hygromycin resistance gene” refer to a hygromycin phosphotransferase gene or gene product.

As used herein, the term “imperfect state” refers to a classification of a fungal organism having no demonstrable sexual life stage.

The term “inhibitor,” as used herein, refers to a chemical substance that eliminates or substantially reduces the activity of ALS catalytic subunit, ALS regulatory subunit, or ALAS holoenzyme, wherein “substantially” means a reduction at least as great as the standard deviation for a measurement, preferably a reduction to 50% activity, more preferably a reduction of at least one magnitude, i.e. to 10% activity. The inhibitor may function by interacting directly with the polypeptide, a cofactor of the polypeptide or any combination thereof.

A polynucleotide may be “introduced” into a fungal cell by any means known to those of skill in the art, including transfection, transformation or transduction, transposable element, electroporation, particle bombardment, infection, and the like. The introduced polynucleotide may be maintained in the cell stably if it is incorporated into a non-chromosomal autonomous replicon or integrated into the fungal chromosome. Alternatively, the introduced polynucleotide may be present on an extra-chromosomal non-replicating vector and be transiently expressed or transiently active.

As used herein, the term “knockout” or “gene disruption” refers to the creation of organisms carrying a null mutation (a mutation in which there is no active gene product), a partial null mutation or mutations, or an alteration or alterations in gene regulation by interrupting a DNA sequence through insertion of a foreign piece of DNA. Usually the foreign DNA encodes a selectable marker.

As used herein, the term “mutant form” of a gene refers to a gene that has been altered, either naturally or artificially, by changing the base sequence of the gene. The change in the base sequence may be of several different types, including changes of one or more bases for different bases, deletions, and/or insertions, such as by a transposon. In contrast, a normal form of a gene (wild-type) is a form commonly found in natural populations of an organism. Commonly a single form of a gene will predominate in natural populations. In general, such a gene is suitable as a normal form of a gene; however, other forms that provide similar functional characteristics may also be used as a normal gene. In particular, a normal form of a gene does not confer a growth conditional phenotype on the strain having that gene, while a mutant form of a gene suitable for use in these methods does provide such a growth conditional phenotype.

As used herein, the term “Ni-NTA” refers to nickel sepharose.

As used herein, a “normal” form of a gene (wild-type) is a form commonly found in natural populations of an organism. Commonly a single form of a gene will predominate in natural populations. In general, such a gene is suitable as a normal form of a gene; however, other forms that provide similar functional characteristics may also be used as a normal gene. In particular, a normal form of a gene does not confer a growth conditional phenotype on the strain having that gene, while a mutant form of a gene suitable for use in these methods does provide such a growth conditional phenotype.

As used herein, the term “pathogenicity” refers to a capability of causing disease and/or degree of capacity to cause disease. The term is applied to parasitic microorganisms in relation to their hosts. As used herein, “pathogenicity,” “pathogenic,” and the like, encompass the general capability of causing disease as well as various mechanisms and structural and/or functional deviations from normal used in the art to describe the causative factors and/or mechanisms, presence, pathology, and/or progress of disease, such as virulence, host recognition, cell wall degradation, toxin production, infection hyphae, penetration peg production, appressorium production, lesion formation, sporulation, and the like.

The “percent (%) sequence identity” between two polynucleotide or two polypeptide sequences is determined according to either the BLAST program (Basic Local Alignment Search Tool, (Altschul, S. F. et al., 215 J. Mol. Biol. 403 (1990) (PMID: 2231712)) or using Smith Waterman Alignment (T. F. Smith & M. S. Waterman 147 J. Mol. Biol. 195 (1981) (PMID: 7265238)). It is understood that for the purposes of determining sequence identity when comparing a DNA sequence to an RNA sequence, a thymine nucleotide is equivalent to a uracil nucleotide.

By “polypeptide” is meant a chain of at least two amino acids joined by peptide bonds. The chain may be linear, branched, circular or combinations thereof. The polypeptides may contain amino acid analogs and other modifications, including, but not limited to glycosylated or phosphorylated residues.

As used herein, the term “proliferation” is synonymous to the term “growth.”

As used herein, “semi-permissive conditions” are conditions in which the relevant culture parameter for a particular growth conditional phenotype is intermediate between permissive conditions and non-permissive conditions. Consequently, in semi-permissive conditions an organism having a growth conditional phenotype will exhibit growth rates intermediate between those shown in permissive conditions and non-permissive conditions. In general, such intermediate growth rate may be due to a mutant cellular component that is partially functional under semi-permissive conditions, essentially fully functional under permissive conditions, and is non-functional or has very low function under non-permissive conditions, where the level of function of that component is related to the growth rate of the organism. An intermediate growth rate may also be a result of a nutrient substance or substances that are present in amounts not sufficient for optimal growth rates to be achieved.

“Sensitivity phenotype” refers to a phenotype that exhibits either hypersensitivity or hyposensitivity.

The term “specific binding” refers to an interaction between a molecule or compound and ALS catalytic subunit, ALS regulatory subunit or ALS holoenzyme, wherein the interaction is dependent upon the primary amino acid sequence and/or the tertiary conformation of ALS catalytic subunit, ALS regulatory subunit or ALS holo enzyme.

“Transform,” as used herein, refers to the introduction of a polynucleotide (single or double stranded DNA, RNA, or a combination thereof) into a living cell by any means. Transformation may be accomplished by a variety of methods, including, but not limited to, electroporation, polyethylene glycol mediated uptake, particle bombardment, agrotransformation, and the like. The transformation process may result in transient or stable expression of the transformed polynucleotide. By “stably transformed” is meant that the sequence of interest is integrated into a replicon in the cell, such as a chromosome or episome. Transformed cells encompass not only the end product of a transformation process, but also the progeny thereof, which retain the polynucleotide of interest.

For the purposes of the invention, “transgenic” refers to any cell, spore, tissue or part that contains all or part of at least one recombinant polynucleotide. In many cases, all or part of the recombinant polynucleotide is stably integrated into a chromosome or stable extra-chromosomal element, so that it is passed on to successive generations.

As used herein, the term “Tween 20” means sorbitan mono-9-octadecenoate poly(oxy-1,1-ethanediyl).

As used in this disclosure, the term “viability” of an organism refers to the ability of an organism to demonstrate growth under conditions appropriate for the organism, or to demonstrate an active cellular function. Some examples of active cellular functions include respiration as measured by gas evolution, secretion of proteins and/or other compounds, dye exclusion, mobility, dye oxidation, dye reduction, pigment production, changes in medium acidity, and the like.

The present inventors have discovered that disruption of either Magnaporthe grisea ILV2 gene encoding an ALS catalytic subunit or ILV6 gene encoding an ALS regulatory subunit severely reduces the growth and pathogenicity of the fungus. Thus, the inventors demonstrate that ALS enzyme, as well as, either of the ALS catalytic or regulatory subunits alone, is a target for antibiotics, preferably fungicides. The activity of yeast putative ALS catalytic and regulatory subunit proteins has been previously studied (Pang S. S., and Duggleby R. G. 38 Biochemistry, 5222-31 (1999); herein incorporated in its entirety by reference; Pang S. S., and Duggleby R. G. 357 The Biochemical Journal, 749-57 (2001); herein incorporated in its entirety by reference). In these studies it was demonstrated that the yeast ALS regulatory subunit protein stimulates the catalytic activity of the catalytic subunit of yeast ALS by up to 7-fold and confers upon it sensitivity to inhibition by valine to levels equivalent to the catalytic subunit alone.

Accordingly, the invention provides methods for identifying compounds that inhibit ALS gene expression or ALS catalytic activity. Such methods include ligand binding assays, assays for enzyme activity, cell-based assays, and assays for ALS gene expression. The compounds identified by the methods of the invention are useful as antibiotics.

Thus, in one embodiment, the invention provides a method for identifying a test compound as a candidate for an antibiotic, comprising contacting an ALS catalytic subunit polypeptide, an ALS regulatory subunit polypeptide or both an ALS catalytic subunit polypeptide and an ALS regulatory subunit polypeptide with a test compound; and detecting the presence or absence of binding between the test compound and the ALS polypeptide, wherein binding indicates that the test compound is a candidate for an antibiotic.

ALS catalytic subunit polypeptides of the invention have the amino acid sequence of a naturally occurring ALS catalytic subunit found in a fungus, animal, plant, or microorganism, or have an amino acid sequence derived from a naturally occurring sequence. Preferably the ALS catalytic subunit is a fungal ALS catalytic subunit. A cDNA encoding M. grisea ALS catalytic subunit protein is set forth in SEQ ID NO:1 and an M. grisea ALS catalytic subunit polypeptide is set forth in SEQ ID NO:2. In one embodiment, the ALS catalytic subunit is a Magnaporthe ALS catalytic subunit. Magnaporthe species include, but are not limited to, Magnaporthe rhizophila, Magnaporthe salvinii, Magnaporthe grisea and Magnaporthe poae and the imperfect states of Magnaporthe in the genus Pyricularia. Preferably, the Magnaporthe ALS catalytic subunit is from Magnaporthe grisea.

In one embodiment, the invention provides a polypeptide consisting essentially of SEQ ID NO:2. For the purposes of the present invention, a polypeptide consisting essentially of SEQ ID NO:2 has at least 90% sequence identity with M. grisea ALS catalytic subunit (SEQ ID NO:2) and at least 10% of the activity of SEQ ID NO:2. A polypeptide consisting essentially of SEQ ID NO:2 has at least 90%, 91%, 92%, 93%, 94%, 95%, 96%, 97%, 98%, or 99% sequence identity with SEQ ID NO:2 and at least 25%, 50%, 75%, or 90% of the activity of M. grisea ALS catalytic subunit. Examples of polypeptides consisting essentially of SEQ ID NO:2 include, but are not limited to, polypeptides having the amino acid sequence of SEQ ID NO:2 with the exception that one or more of the amino acids are substituted with structurally similar amino acids providing a conservative amino acid substitution. Conservative amino acid substitutions are well known to those of skill in the art. Examples of polypeptides consisting essentially of SEQ ID NO:2 include polypeptides having 1, 2, or 3 conservative amino acid substitutions relative to SEQ ID NO:2. Other examples of polypeptides consisting essentially of SEQ ID NO:2 include polypeptides having the sequence of SEQ ID NO:2, but with truncations at either or both the 3′ and the 5′ end. For example, polypeptides consisting essentially of SEQ ID NO:2 include polypeptides having 1, 2, or 3 amino acids residues removed from either or both 3′ and 5′ ends relative to SEQ ID NO:2.

ALS regulatory subunit polypeptides of the invention have the amino acid sequence of a naturally occurring ALS regulatory subunit found in a fungus, animal, plant, or microorganism, or have an amino acid sequence derived from a naturally occurring sequence. Preferably the ALS regulatory subunit is a fungal ALS regulatory subunit. A cDNA encoding M. grisea ALS regulatory subunit protein is set forth in SEQ ID NO:3, an M. grisea ALS regulatory subunit genomic DNA is set forth in SEQ ID NO:4, and an M. grisea ALS regulatory subunit polypeptide is set forth in SEQ ID NO:5. In one embodiment, the ALS regulatory subunit is a Magnaporthe ALS regulatory subunit. Magnaporthe species include, but are not limited to, Magnaporthe rhizophila, Magnaporthe salvinii, Magnaporthe grisea and Magnaporthe poae and the imperfect states of Magnaporthe in the genus Pyricularia. Preferably, the Magnaporthe ALS regulatory subunit is from Magnaporthe grisea.

In one embodiment, the invention provides a polypeptide consisting essentially of SEQ ID NO:5. For the purposes of the present invention, a polypeptide consisting essentially of SEQ ID NO:5 has at least 90% sequence identity with M. grisea ALS regulatory subunit (SEQ ID NO:5) and at least 10% of the activity of SEQ ID NO:5. A polypeptide consisting essentially of SEQ ID NO:5 has at least 90%, 91%, 92%, 93%, 94%, 95%, 96%, 97%, 98%, or 99% sequence identity with SEQ ID NO:5 and at least 25%, 50%, 75%, or 90% of the activity of M. grisea ALS regulatory subunit. Examples of polypeptides consisting essentially of SEQ ID NO:5 include, but are not limited to, polypeptides having the amino acid sequence of SEQ ID NO:5 with the exception that one or more of the amino acids are substituted with structurally similar amino acids providing a conservative amino acid substitution. Conservative amino acid substitutions are well known to those of skill in the art. Examples of polypeptides consisting essentially of SEQ ID NO:5 include polypeptides having 1, 2, or 3 conservative amino acid substitutions relative to SEQ ID NO:5. Other examples of polypeptides consisting essentially of SEQ ID NO:5 include polypeptides having the sequence of SEQ ID NO:5, but with truncations at either or both the 3′ and the 5′ end. For example, polypeptides consisting essentially of SEQ ID NO:5 include polypeptides having 1, 2, or 3 amino acids residues removed from either or both 3′ and 5′ ends relative to SEQ ID NO:5.

In various embodiments, the ALS catalytic and/or regulatory subunit can be from Powdery Scab (Spongospora subterranea), Grey Mould (Botrytis cinerea), White Rot (Armillaria mellea), Heartrot Fungus (Ganoderma adspersum), Brown-Rot (Piptoporus betulinus), Corn Smut (Ustilago maydis), Heartrot (Polyporus squamosus), Gray Leaf Spot (Cercospora zeae-maydis), Honey Fungus (Armillaria gallica), Root rot (Armillaria luteobubalina), Shoestring Rot (Armillaria ostoyae), Banana Anthracnose Fungus (Colletotrichum musae), Apple-rotting Fungus (Monilinia fructigena), Apple-rotting Fungus (Penicillium expansum), Clubroot Disease (Plasmodiophora brassicae), Potato Blight (Phytophthora infestans), Root pathogen (Heterobasidion annosum), Take-all Fungus (Gaeumannomyces graminis), Dutch Elm Disease (Ophiostoma ulmi), Bean Rust (Uromyces appendiculatus), Northern Leaf Spot (Cochliobolus carbonum), Milo Disease (Periconia circinata), Southern Corn Blight (Cochliobolus heterostrophus), Leaf Spot (Cochliobolus lunata), Brown Stripe (Cochliobolus stenospilus), Panama disease (Fusarium oxysporum), Wheat Head Scab Fungus (Fusarium graminearum), Cereal Foot Rot (Fusarium culmorum), Potato Black Scurf (Rhizoctonia solani), Wheat Black Stem Rust (Puccinia graminis), White mold (Sclerotinia sclerotiorum), and the like.

Fragments of an ALS catalytic subunit polypeptide are useful in the methods of the invention. In one embodiment, the ALS catalytic subunit fragments include an intact or nearly intact epitope that occurs on the biologically active wild-type ALS catalytic subunit. For example, the fragments comprise at least 10 consecutive amino acids of ALS catalytic subunit set forth in SEQ ID NO:2. The fragments comprise at least 15, 20, 25, 30, 35, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675 or at least 680 consecutive amino acids residues of ALS catalytic subunit set forth in SEQ ID NO:2. Fragments of heterologous ALS catalytic subunits are also useful in the methods of the invention. For example, polypeptides having at least 50%, 60%, 70%, 80%, 90%, 95%, 96%, 97%, 98% or 99% sequence identity with at least 50 consecutive amino acid residues of SEQ ID NO:2 are useful in the methods of the invention. In one embodiment, the fragment is from a Magnaporthe ALS catalytic subunit. In an alternate embodiment, the fragment contains an amino acid sequence conserved among fungal ALS catalytic subunits.

Polypeptides having at least 40% sequence identity with M. grisea ALS catalytic subunit (SEQ ID NO:2) protein are also useful in the methods of the invention. In one embodiment, the sequence identity is at least 40%, 45%, 50%, 55%, 60%, 65%, 70%, 75%, 80%, 85%, 90%, 91%, 92%, 93%, 94%, 95%, 96%, 97%, 98% or 99%, or any integer from 40-100% sequence identity in ascending order with M. grisea ALS catalytic subunit (SEQ ID NO:2) protein. In addition, it is preferred that polypeptides of the invention have at least 10% of the activity of M. grisea ALS catalytic subunit (SEQ ID NO:2) protein. ALS catalytic subunit polypeptides of the invention have at least 10%, 15%, 20%, 25%, 30%, 35%, 40%, 45%, 50%, 55%, 60%, 65%, 70%, 75%, 80%, 85% or at least 90% of the activity of M. grisea ALS catalytic subunit (SEQ ID NO:2) protein.

Fragments of an ALS regulatory subunit polypeptide are useful in the methods of the invention. In one embodiment, the ALS regulatory subunit fragments include an intact or nearly intact epitope that occurs on the biologically active wild-type ALS regulatory subunit. For example, the fragments comprise at least 10 consecutive amino acids of ALS regulatory subunit set forth in SEQ ID NO:5. The fragments comprise at least 15, 20, 25, 30, 35, 40, 50, 60, 70, 80, 90, 100, 125, 150, 175, 200, 225, 250, 275, 300 or at least 315 consecutive amino acids residues of ALS regulatory subunit set forth in SEQ ID NO:5. Fragments of heterologous ALS regulatory subunits are also useful in the methods of the invention. For example, polypeptides having at least 50%, 60%, 70%, 80%, 90%, 95%, 96%, 97%, 98% or 99% sequence identity with at least 50 consecutive amino acid residues of SEQ ID NO:5 are useful in the methods of the invention. In one embodiment, the fragment is from a Magnaporthe ALS regulatory subunit. In an alternate embodiment, the fragment contains an amino acid sequence conserved among fungal ALS regulatory subunits.

Polypeptides having at least 40% sequence identity with M. grisea ALS regulatory subunit (SEQ ID NO:5) protein are also useful in the methods of the invention. In one embodiment, the sequence identity is at least 40%, 45%, 50%, 55%, 60%, 65%, 70%, 75%, 80%, 85%, 90%, 91%, 92%, 93%, 94%, 95%, 96%, 97%, 98% or 99%, or any integer from 40-100% sequence identity in ascending order with M. grisea ALS regulatory subunit (SEQ ID NO:5) protein. In addition, it is preferred that polypeptides of the invention have at least 10% of the activity of M. grisea ALS regulatory subunit (SEQ ID NO:5) protein. ALS regulatory subunit polypeptides of the invention have at least 10%, 15%, 20%, 25%, 30%, 35%, 40%, 45%, 50%, 55%, 60%, 65%, 70%, 75%, 80%, 85% or at least 90% of the activity of M. grisea ALS regulatory subunit (SEQ ID NO:5) protein.

Thus, in another embodiment, the invention provides a method for identifying a test compound as a candidate for an antibiotic, comprising: contacting an ALS polypeptide with a test compound, wherein the ALS polypeptide is selected from the group consisting of: an ALS catalytic subunit polypeptide; an ALS regulatory subunit polypeptide; and an ALS catalytic subunit polypeptide and an ALS regulatory subunit polypeptide; and detecting the presence or absence of binding between the test compound and the ALS polypeptide, wherein binding indicates that the test compound is a candidate for an antibiotic.

In further embodiments of the invention, ALS catalytic subunit polypeptides of the invention include: polypeptides consisting essentially of SEQ ID NO:2; polypeptides having at least ten consecutive amino acids of SEQ ID NO:2; polypeptides having at least 50% sequence identity with SEQ ID NO:2 and at least 10% of the activity of SEQ ID NO:2; and polypeptides consisting of at least 50 amino acids having at least 50% sequence identity with SEQ ID NO:2 and at least 10% of the activity of SEQ ID NO:2. ALS regulatory subunit polypeptides of the invention include: polypeptides consisting essentially of SEQ ID NO:5; polypeptides having at least ten consecutive amino acids of SEQ ID NO:5; polypeptides having at least 50% sequence identity with SEQ ID NO:5 and at least 10% of the activity of SEQ ID NO:5; and polypeptides consisting of at least 50 amino acids having at least 50% sequence identity with SEQ ID NO:5 and at least 10% of the activity of SEQ ID NO:5.

Any technique for detecting the binding of a ligand to its target may be used in the methods of the invention. For example, the ligand and target are combined in a buffer. Many methods for detecting the binding of a ligand to its target are known in the art, and include, but are not limited to, the detection of an immobilized ligand-target complex or the detection of a change in the properties of a target when it is bound to a ligand. For example, in one embodiment, an array of immobilized candidate ligands is provided. The immobilized ligands are contacted with an ALS catalytic subunit polypeptide, an ALS regulatory subunit polypeptide, or an ALS catalytic and an ALS regulatory subunit polypeptide, or a fragment or variant thereof, the unbound polypeptide is removed and the bound ALS polypeptide is detected. In a preferred embodiment, bound ALS polypeptide is detected using a labeled binding partner, such as a labeled antibody. In an alternate preferred embodiment, ALS polypeptide is labeled prior to contacting the immobilized candidate ligands. Preferred labels include fluorescent or radioactive moieties. Preferred detection methods include fluorescence correlation spectroscopy (FCS) and FCS-related confocal nanofluorimetric methods.

Once a compound is identified as a candidate for an antibiotic using a binding assay, it is tested for ability to inhibit ALS polypeptide activity. The compound is tested using either in vitro or cell based assays. Alternatively, a compound can be tested by applying it directly to a fungus or fungal cell, or expressing it therein, and monitoring the fungus or fungal cell for changes or decreases in growth, development, viability, pathogenicity, or alterations in gene expression. Thus, in one embodiment, the invention provides a method for determining whether a compound identified as an antibiotic candidate by an above method has antifungal activity, further comprising: contacting a fungus or fungal cells with the antifungal candidate and detecting a decrease in the growth, viability, or pathogenicity of the fungus or fungal cells.

By decrease in growth, is meant that the antifungal candidate causes at least a 10% decrease in the growth of the fungus or fungal cells, as compared to the growth of the fungus or fungal cells in the absence of the antifungal candidate. By a decrease in viability is meant that at least 20% of the fungal cells, or portion of the fungus contacted with the antifungal candidate are nonviable. Preferably, the growth or viability will be decreased by at least 40%. More preferably, the growth or viability will be decreased by at least 50%, 75% or at least 90% or more. Methods for measuring fungal growth and cell viability are known to those skilled in the art. By decrease in pathogenicity, is meant that the antifungal candidate causes at least a 10% decrease in the disease caused by contact of the fungal pathogen with its host, as compared to the disease caused in the absence of the antifungal candidate. Preferably, the disease will be decreased by at least 40%. More preferably, the disease will be decreased by at least 50%, 75% or at least 90% or more. Methods for measuring fungal disease are well known to those skilled in the art, and include such metrics as lesion formation, lesion size, sporulation, respiratory failure, and/or death.

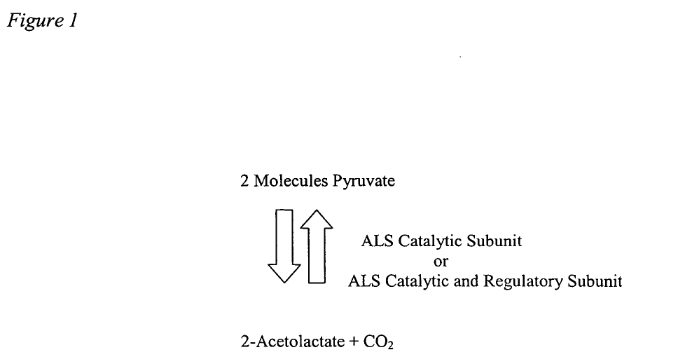

The ability of a compound to inhibit ALS polypeptide activity can be detected using in vitro enzymatic assays in which the disappearance of a substrate or the appearance of a product is directly or indirectly detected. The ALS catalytic subunit catalyzes the interconversion of two pyruvate molecules with 2-acetolactate and CO₂ (FIG. 1) at a rate lower than that achieved in the presence of the ALS regulatory subunit in the absence of amino acids. An ALS regulatory subunit is a polypeptide capable of increasing enzymatic activity of an ALS catalytic subunit in the absence of amino acids. Therefore, methods for measuring the ability of a test compound to inhibit activity of ALS catalytic or regulatory subunit activity include detecting the effect of the presence of the compound on the progression of ALS enzymatic interconversion of two pyruvate molecules with 2-acetolactate and CO₂. Suitable reaction conditions and buffers for measuring enzymatic activity in general, and ALS activity in particular, are well known to those of ordinary skill in the art. See Pang, S. S., and Duggleby R. G., supra, for an example of methods for measuring ALS activity.

The methods of the invention encompass several enzymatic assays for the identification of inhibitors of ALS activity. In one embodiment of the invention, the enzymatic assay is designed to identify inhibitors that are specific for the ALS catalytic subunit. In another embodiment, inhibitors that specifically target the function of the regulatory subunit are identified. In a third embodiment, compounds are identified that inhibit the activity of the ALS catalytic/regulatory complex. For example, one method for identifying a compound as a candidate for an antibiotic, based on ability to inhibit ALS catalytic subunit activity comprises: (a) contacting an ALS catalytic subunit with a suitable reaction mixture comprising pyruvate in the presence and absence of a test compound; and (b) comparing the concentration of any one or more of the individual reactants, pyruvate, 2-acetolactate and CO₂ in step (a). A change in the concentration of any one or more of the reactants in the presence, relative to the absence, of the compound indicates the compound as a candidate for an antibiotic.

In another embodiment, a method for identifying a compound as a candidate for an antibiotic, based on ability to inhibit ALS regulatory subunit activity comprises: (a) contacting an ALS regulatory subunit and an ALS catalytic subunit (ALS catalytic/regulatory subunit complex) with a suitable reaction mixture comprising pyruvate in the presence and absence of a test compound; (b) contacting the ALS catalytic subunit alone with the reaction mixture comprising pyruvate in the presence and absence of the test compound; and (c) comparing the concentration of any one or more of the individual reactants, pyruvate, 2-acetolactate and CO₂ in each of steps (a) and (b). A change in concentration of any one or more of the reactants in the presence, relative to the absence, of the compound in step (a) and no change in the concentration of reactants in step (b) indicates the compound as a candidate for an antibiotic.

In a third embodiment, a method for identifying a compound as a candidate for an antibiotic, based on ability to inhibit ALS catalytic/regulatory subunit complex activity comprises: (a) contacting an ALS regulatory subunit and an ALS catalytic subunit (ALS catalytic/regulatory subunit complex) with a suitable reaction mixture comprising pyruvate in the presence and absence of a test compound; and (b) comparing the concentration of any one or more of the individual reactants, pyruvate, 2-acetolactate and CO₂ in step (a). A change in the concentration of any one or more of the reactants in the presence, relative to the absence, of the compound indicates the compound as a candidate for an antibiotic. In each of the three embodiments described above, direct or indirect detection of any one or more of the individual reactants, pyruvate, 2-acetolactate and CO₂, is performed using any of the methods commonly known to one of ordinary skill in the art including, spectrophotometry, fluorimetry, mass spectroscopy, thin layer chromatography (TLC) and reverse phase HPLC.

In a particular embodiment of the invention, the ALS catalytic subunit is SEQ ID NO:2. In another embodiment of the invention, the ALS regulatory subunit is SEQ ID NO:5. In another embodiment of the invention, the ALS regulatory subunit is SEQ ID NO:5 and the ALS catalytic subunit is SEQ ID NO:2. In another embodiment of the invention, the ALS regulatory subunit and ALS catalytic subunit are M. grisea polypeptides. In another embodiment of the invention, the ALS regulatory subunit and the ALS catalytic subunit are fungal polypeptides. In another embodiment of the invention the ALS regulatory subunit and ALS catalytic subunit are plant pathogenic fungal polypeptides. In another embodiment of the invention the ALS regulatory subunit and ALS catalytic subunit are animal pathogenic fungal polypeptides. In the methods of the invention, the ALS regulatory subunit and ALS catalytic subunit are generally derived from the same organism, although a common origin is not a requirement of the invention.

Polypeptides consisting essentially of SEQ ID NO:2 and SEQ ID NO:5; active polypeptide fragments of ALS catalytic and regulatory subunit polypeptides; and heterologous ALS catalytic and regulatory subunit polypeptides, and fragments thereof, and are also useful in the methods of the invention. In one embodiment of the invention, the ALS catalytic subunit is a polypeptide consisting essentially of SEQ ID NO:2. In another embodiment of the invention, the ALS regulatory subunit is a polypeptide consisting essentially of SEQ ID NO:5. In another embodiment of the invention, the ALS catalytic subunit is a polypeptide consisting essentially of SEQ ID NO:2 and the ALS regulatory subunit is a polypeptide consisting essentially of SEQ ID NO:5.

In another embodiment of the invention, the ALS catalytic subunit is a polypeptide comprising at least 50 consecutive amino acid residues and at least 10% of the activity of SEQ ID NO:2. In another embodiment of the invention, the ALS regulatory subunit is a polypeptide comprising at least 50 consecutive amino acid residues and at least 10% of the activity of SEQ ID NO:5. In another embodiment of the invention, the ALS catalytic subunit is a polypeptide comprising at least 50 consecutive amino acid residues and at least 10% of the activity of SEQ ID NO:2 and the ALS regulatory subunit is a polypeptide comprising at least 50 consecutive amino acid residues and at least 10% of the activity of SEQ ID NO:5.

In another embodiment of the invention, the ALS catalytic subunit is a polypeptide having at least 10% of the activity of SEQ ID NO:2 and at least 50%, 60%, 70%, 80%, 90%, 95%, 96%, 97%, 98% or 99% sequence identity with SEQ ID NO:2. In another embodiment of the invention, the ALS regulatory subunit is a polypeptide having at least 10% of the activity of SEQ ID NO:5 and at least 50%, 60%, 70%, 80%, 90%, 95%, 96%, 97%, 98% or 99% sequence identity with SEQ ID NO:5. In another embodiment of the invention, the ALS catalytic subunit is a polypeptide having at least 10% of the activity of SEQ ID NO:2 and at least 50%, 60%, 70%, 80%, 90%, 95%, 96%, 97%, 98% or 99% sequence identity with SEQ ID NO:2 and the ALS regulatory subunit is a polypeptide having at least 10% of the activity of SEQ ID NO:5 and at least 50%, 60%, 70%, 80%, 90%, 95%, 96%, 97%, 98% or 99% sequence identity with SEQ ID NO:5. Most preferably, the ALS catalytic and/or regulatory polypeptide has at least 50% sequence identity with SEQ ID NO:2 and/or SEQ ID NO:5, respectively, and at least 25%, 75% or at least 90% of the activity thereof.

In another embodiment of the invention, the ALS catalytic subunit is a polypeptide having at least 10% of the activity of SEQ ID NO:2 and at least 50%, 60%, 70%, 80%, 90%, 95%, 96%, 97%, 98% or 99% sequence identity with at least 50 consecutive amino acid residues of SEQ ID NO:2. In another embodiment of the invention, the ALS regulatory subunit is a polypeptide having at least 10% of the activity of SEQ ID NO:5 and at least 50%, 60%, 70%, 80%, 90%, 95%, 96%, 97%, 98% or 99% sequence identity with at least 50 consecutive amino acid residues of SEQ ID NO:5. In another embodiment of the invention, the ALS catalytic subunit is a polypeptide having at least 10% of the activity of SEQ ID NO:2 and at least 50%, 60%, 70%, 80%, 90%, 95%, 96%, 97%, 98% or 99% sequence identity with at least 50 consecutive amino acid residues of SEQ ID NO:2 and the ALS regulatory subunit is a polypeptide having at least 10% of the activity of SEQ ID NO:5 and at least 50%, 60%, 70%, 80%, 90%, 95%, 96%, 97%, 98% or 99% sequence identity with at least 50 consecutive amino acid residues of SEQ ID NO:5. Most preferably, the ALS catalytic and/or regulatory polypeptide has at least 50% sequence identity with at least 50 consecutive amino acid residues of SEQ ID NO:2 and/or SEQ ID NO:5, respectively, and at least 25%, 75% or at least 90% of the activity thereof.

For in vitro assays, ALS regulatory and catalytic subunit proteins and derivatives thereof may be isolated from a fungus or may be recombinantly produced in and isolated from an archael, bacterial, fungal, or other eukaryotic cell culture. Preferably the polypeptides are produced using an E. coli, yeast, or filamentous fingal expression system. Methods, known to those of ordinary skill in the art, for the isolation of polypeptides are useful for the isolation of the ALS regulatory and catalytic subunit proteins of the invention. See Pang, S. S. and Duggleby R. G., supra, for an example of methods for isolating ALS regulatory and catalytic subunit polypeptides.

As an alternative to in vitro assays, the invention also provides cell-based assays. In one embodiment, the invention provides a method for identifying a test compound as a candidate for an antibiotic, comprising: a) measuring the expression or activity of an ALS catalytic and/or regulatory subunit in a cell, cells, tissue, or an organism in the absence of a test compound; b) contacting the cell, cells, tissue, or organism with the test compound and measuring the expression or activity of the ALS catalytic and/or regulatory subunit in the cell, cells, tissue, or organism; and c) comparing the expression or activity of the ALS catalytic and/or regulatory subunit in steps (a) and (b), wherein an altered expression or activity in the presence of the test compound indicates that the compound is a candidate for an antibiotic.

Expression of an ALS catalytic or regulatory subunit can be measured by detecting the ALS catalytic or regulatory subunit primary transcript or mRNA, ALS catalytic or regulatory subunit polypeptide, or ALS catalytic or regulatory subunit enzymatic activity. Methods for detecting the expression of RNA and proteins are known to those skilled in the art. (Current Protocols in Molecular Biology, Ausubel et al., eds., Greene Publishing & Wiley-Interscience, New York, (1995)). The method of detection is not critical to the present invention. Methods for detecting ALS catalytic or regulatory subunit RNA include, but are not limited to, amplification assays such as quantitative reverse transcriptase-PCR, and/or hybridization assays such as Northern analysis, dot blots, slot blots, in-situ hybridization, transcriptional fusions using an ALS catalytic or regulatory subunit promoter fused to a reporter gene, DNA assays, and microarray assays.

Methods for detecting protein expression include, but are not limited to, immunodetection methods such as Western blots, ELISA assays, polyacrylamide gel electrophoresis, mass spectroscopy, and enzymatic assays. Also, any reporter gene system may be used to detect ALS catalytic or regulatory subunit protein expression. For detection using gene reporter systems, a polynucleotide encoding a reporter protein is fused in frame with ALS catalytic or regulatory subunit so as to produce a chimeric polypeptide. Methods for using reporter systems are known to those skilled in the art.

Chemicals, compounds, or compositions identified by the above methods as modulators of ALS catalytic and/or regulatory subunit expression or activity are useful for controling fungal growth. Diseases such as rusts, mildews, and blights spread rapidly once established. Fungicides are thus routinely applied to growing and stored crops as a preventive measure, generally as foliar sprays or seed dressings. For example, compounds that inhibit fungal growth can be applied to a fungus or expressed in a fungus to prevent fungal growth. Thus, the invention provides a method for inhibiting fungal growth, comprising contacting a fungus with a compound identified by the methods of the invention as having antifungal activity.

Antifungals and antifungal inhibitor candidates identified by the methods of the invention can be used to control the growth of undesired fungi, including ascomycota, zygomycota, basidiomycota, chytridiomycota, and lichens. Examples of undesired fungi include, but are not limited to Powdery Scab (Spongospora subterranea), Grey Mould (Botrytis cinerea), White Rot (Armillaria mellea), Heartrot Fungus (Ganoderma adspersum), Brown-Rot (Piptoporus betulinus), Corn Smut (Ustilago maydis), Heartrot (Polyporus squamosus), Gray Leaf Spot (Cercospora zeae-maydis), Honey Fungus (Armillaria gallica), Root rot (Armillaria luteobubalina), Shoestring Rot (Armillaria ostoyae), Banana Anthracnose Fungus (Colletotrichum musae), Apple-rotting Fungus (Monilinia fructigena), Apple-rotting Fungus (Penicillium expansum), Clubroot Disease (Plasmodiophora brassicae), Potato Blight (Phytophthora infestans), Root pathogen (Heterobasidion annosum), Take-all Fungus (Gaeumannomyces graminis), Dutch Elm Disease (Ophiostoma ulmi), Bean Rust (Uromyces appendiculatus), Northern Leaf Spot (Cochliobolus carbonum), Milo Disease (Periconia circinata), Southern Corn Blight (Cochliobolus heterostrophus), Leaf Spot (Cochliobolus lunata), Brown Stripe (Cochliobolus stenospilus), Panama disease (Fusarium oxysporum), Wheat Head Scab Fungus (Fusarium graminearum), Cereal Foot Rot (Fusarium culmorum), Potato Black Scurf (Rhizoctonia solani), Wheat Black Stem Rust (Puccinia graminis), White mold (Sclerotinia sclerotiorum), diseases of animals such as infections of lungs, blood, brain, skin, scalp, nails or other tissues (Aspergillus fumigatus Aspergillus sp. Fusraium sp., Trichophyton sp., Epidermophyton sp., and Microsporum sp., and the like).

Also provided in the invention are methods of screening for an antibiotic by determining the in vivo activity of a test compound against two separate fungal organisms, wherein the fingal organisms comprise a first form of an ALS catalytic subunit and a second form of the ALS catalytic subunit, respectively. In the methods of the invention, at least one of the two forms of the ALS catalytic subunit has at least 10% of the activity of the polypeptide set forth in SEQ ID NO:2. The methods comprise comparing the growth of the two organisms in the presence of the test compound relative to their respective controls without the test compound. A difference in growth between the two organisms in the presence of the test compound indicates that the test compound is a candidate for an antibiotic.

Forms of an ALS catalytic subunit useful in the methods of the invention are selected from the group consisting of: a nucleic acid encoding SEQ ID NO:2; a nucleic acid encoding a polypeptide consisting essentially of SEQ ID NO:2; a nucleic acid set forth in SEQ ID NO:1; a nucleic acid set forth in SEQ ID NO:1 comprising a mutation either reducing or abolishing ALS catalytic subunit protein activity; a nucleic acid encoding a heterologous ALS catalytic subunit; and a nucleic acid set encoding a heterologous ALS catalytic subunit comprising a mutation either reducing or abolishing ALS catalytic subunit protein activity. Any combination of two different forms of the ALS catalytic subunit genes listed above are useful in the methods of the invention, with the caveat that at least one of the forms of the ALS catalytic subunit has at least 10% of the activity of the polypeptide set forth in SEQ ID NO:2.

Also provided in the invention are methods of screening for an antibiotic by determining the in vivo activity of a test compound against two separate fungal organisms, wherein the fungal organisms comprise a first form of an ALS regulatory subunit and a second form of the ALS regulatory subunit, respectively. In the methods of the invention, at least one of the two forms of the ALS regulatory subunit has at least 10% of the activity of the polypeptide set forth in SEQ ID NO:5. The methods comprise comparing the growth of the two organisms in the presence of the test compound relative to their respective controls without the test compound. A difference in growth between the two organisms in the presence of the test compound indicates that the test compound is a candidate for an antibiotic.

Forms of an ALS regulatory subunit useful in the methods of the invention are selected from the group consisting of: a nucleic acid encoding SEQ ID NO:5; a nucleic acid encoding a polypeptide consisting essentially of SEQ ID NO:5; a nucleic acid set forth in SEQ ID NO:3 or SEQ ID NO:4; a nucleic acid set forth in SEQ ID NO:3 or SEQ ID NO:4 comprising a mutation either reducing or abolishing ALS regulatory subunit protein activity; a nucleic acid encoding a heterologous ALS regulatory subunit; and a nucleic acid encoding a heterologous ALS regulatory subunit comprising a mutation either reducing or abolishing ALS regulatory subunit protein activity. Any combination of two different forms of the ALS regulatory subunit genes listed above are useful in the methods of the invention, with the caveat that at least one of the forms of the ALS regulatory subunit has at least 10% of the activity of the polypeptide set forth in SEQ ID NO:5.

Thus, in one embodiment, the invention provides a method for identifying a test compound as a candidate for an antibiotic, comprising: providing an organism having a first form of an ALS catalytic or regulatory subunit; providing an organism having a second form of the ALS catalytic or regulatory subunit; and determining the growth of the organism having the first form of the ALS catalytic or regulatory subunit and the growth of the organism having the second form of the ALS catalytic or regulatory subunit in the presence of the test compound, wherein a difference in growth between the two organisms in the presence of the test compound indicates that the test compound is a candidate for an antibiotic. It is recognized in the art that the optional determination of the growth of the organism having the first form of the ALS catalytic or regulatory subunit and the growth of the organism having the second form of the ALS catalytic or regulatory subunit in the absence of any test compounds is performed to control for any inherent differences in growth as a result of the different genes. Growth and/or proliferation of an organism are measured by methods well known in the art such as optical density measurements, and the like. In a preferred embodiment, the organism is Magnaporthe grisea.

In another embodiment, the invention provides a method for identifying a test compound as a candidate for an antibiotic, comprising: providing an organism having a first form of an ALS catalytic or regulatory subunit; providing a comparison organism having a second form of the ALS catalytic or regulatory subunit; and determining the pathogenicity of the organism having the first form of the ALS catalytic or regulatory subunit and the organism having the second form of the ALS catalytic or regulatory subunit in the presence of the test compound, wherein a difference in pathogenicity between the two organisms in the presence of the test compound indicates that the test compound is a candidate for an antibiotic. In an alternate embodiment of the inventon, the pathogenicity of the organism having the first form of the ALS catalytic or regulatory subunit and the organism having the second form of the ALS catalytic or regulatory subunit in the absence of any test compounds is determined to control for any inherent differences in pathogenicity as a result of the different genes. Pathogenicity of an organism is measured by methods well known in the art such as lesion number, lesion size, sporulation, and the like. In a preferred embodiment the organism is Magnaporthe grisea.

In one embodiment of the invention, the first form of an ALS catalytic subunit is SEQ ID NO:1 and the second form of the ALS catalytic subunit is a ALS catalytic subunit that confers a growth conditional phenotype (i.e. a branched chain amino acid requiring phenotype) and/or a hypersensitivity or hyposensitivity phenotype on the organism. In a related embodiment of the invention, the second form of the ALS catalytic subunit is SEQ ID NO:1 comprising a transposon insertion that reduces activity. In still another embodiment of the invention, the second form of a ALS catalytic subunit is SEQ ID NO:1 comprising a transposon insertion that abolishes activity. In yet another embodiment of the invention, the second form of the ALS catalytic subunit is N. crassa ALS catalytic subunit. In a related embodiment of the invention, the second form of the ALS catalytic subunit is Saccharomyces cerevisiea ALS catalytic subunit.

In one embodiment of the invention, the first form of an ALS regulatory subunit is SEQ ID NO:3 or SEQ ID NO:4, and the second form of the ALS regulatory subunit is a ALS regulatory subunit that confers a growth conditional phenotype (i.e. a branched chain amino acid requiring phenotype) and/or a hypersensitivity or hyposensitivity phenotype on the organism. In a related embodiment of the invention, the second form of the ALS regulatory subunit is SEQ ID NO:3 comprising a transposon insertion that reduces activity. In still another embodiment of the invention, the second form of a ALS regulatory subunit is SEQ ID NO:3 comprising a transposon insertion that abolishes activity. In a related embodiment of the invention, the second form of the ALS regulatory subunit is SEQ ID NO:4 comprising a transposon insertion that reduces activity. In a further embodiment of the invention, the second form of the ALS regulatory subunit is SEQ ID NO:4 comprising a transposon insertion that abolishes activity. In yet another embodiment of the invention, the second form of the ALS regulatory subunit is N. crassa ALS regulatory subunit. In a related embodiment of the invention, the second form of the ALS regulatory subunit is Saccharomyces cerevisiea ALS regulatory subunit.

In another embodiment of the invention, the first form of an ALS catalytic or regulatory subunit is N. crassa ALS catalytic or regulatory subunit and the second form of the ALS catalytic or regulatory subunit is N. crassa ALS catalytic or regulatory subunit comprising a transposon insertion that reduces activity. In a related embodiment of the invention, the second form of the ALS catalytic or regulatory subunit is N. crassa ALS catalytic or regulatory subunit comprising a transposon insertion that abolishes activity. In another embodiment of the invention, the first form of an ALS catalytic or regulatory subunit is Saccharomyces cerevisiea ALS catalytic or regulatory subunit and the second form of the ALS catalytic or regulatory subunit is Saccharomyces cerevisiea ALS catalytic or regulatory subunit comprising a transposon insertion that reduces activity. In a related embodiment of the invention, the second form of the ALS catalytic or regulatory subunit is Saccharomyces cerevisiea ALS catalytic or regulatory subunit comprising a transposon insertion that abolishes activity.

Conditional lethal mutants and/or antipathogenic mutants identify particular biochemical and/or genetic pathways given that at least one identified target gene is present in that pathway. Knowledge of these pathways allows for the screening of test compounds as candidates for antibiotics as inhibitors of the substrates, products, proteins and/or enzymes of the pathway. The invention provides methods of screening for an antibiotic by determining whether a test compound is active against the branched chain amino acid biosynthetic pathway on which ALS catalytic and regulatory subunit functions. Pathways known in the art are found at the Kyoto Encyclopedia of Genes and Genomes and in standard biochemistry texts (See, e.g. Lehninger et al., Principles of Biochemistry, New York, Worth Publishers (1993)).

Thus, in one embodiment, the invention provides a method for screening for test compounds acting against the biochemical and/or genetic pathway or pathways in which ALS catalytic and regulatory subunit functions, comprising: providing an organism having a first form of a gene in the branched chain amino acid biosynthetic pathway; providing an organism having a second form of the gene in the branched chain amino acid biosynthetic pathway; and determining the growth of the two organisms in the presence of a test compound, wherein a difference in growth between the organism having the first form of the gene and the organism having the second form of the gene in the presence of the test compound indicates that the test compound is a candidate for an antibiotic. It is recognized in the art that the optional determination of the growth of the organism having the first form of the gene and the organism having the second form of the gene in the absence of any test compounds is performed to control for any inherent differences in growth as a result of the different genes. Growth and/or proliferation of an organism are measured by methods well known in the art, such as optical density measurements and the like. In a preferred embodiment, the organism is Magnaporthe grisea.

The forms of a gene in the branched chain amino acid biosynthetic pathway useful in the methods of the invention include, for example, wild-type and mutated genes encoding ketol-acid reductoisomerase and dihydroxy-acid dehydratase from any organism, preferably from a fungal organism, and most preferrably from M. grisea. The forms of a mutated gene in the branched chain amino acid biosynthetic pathway comprise a mutation either reducing or abolishing protein activity. In one example, the form of a gene in the branched chain amino acid biosynthetic pathway comprises a transposon insertion. Any combination of a first form of a gene in the branched chain amino acid biosynthetic pathway and a second form of the gene listed above are useful in the methods of the invention, with the limitation that one of the forms of a gene in the branched chain amino acid biosynthetic pathway has at least 10% of the activity of the corresponding M. grisea gene.

In another embodiment, the invention provides a method for screening for test compounds acting against the biochemical and/or genetic pathway or pathways in which ALS catalytic and regulatory subunit functions, comprising: providing an organism having a first form of a gene in the branched chain amino acid biosynthetic pathway; providing an organism having a second form of the gene in the branched chain amino acid biosynthetic pathway; and determining the pathogenicity of the two organisms in the presence of the test compound, wherein a difference in pathogenicity between the organism having the first form of the gene and the organism having the second form of the gene in the presence of the test compound indicates that the test compound is a candidate for an antibiotic. In an optional embodiment of the inventon, the pathogenicity of the two organisms in the absence of any test compounds is determined to control for any inherent differences in pathogenicity as a result of the different genes. Pathogenicity of an organism is measured by methods well known in the art such as lesion number, lesion size, sporulation, and the like. In a preferred embodiment the organism is Magnaporthe grisea.

Thus, in an alternate embodiment, the invention provides a method for screening for test compounds acting against the biochemical and/or genetic pathway or pathways in which ALS catalytic and regulatory subunit functions, comprising: providing paired growth media containing a test compound, wherein the paired growth media comprise a first medium and a second medium and the second medium contains a higher level of one or more branched chain amino acids than the first medium; inoculating the first and the second medium with an organism; and determining the growth of the organism, wherein a difference in growth of the organism between the first and the second medium indicates that the test compound is a candidate for an antibiotic. In one embodiment of the invention, the growth of the organism is determined in the first and the second medium in the absence of any test compounds to control for any inherent differences in growth as a result of the different media. Growth and/or proliferation of the organism are measured by methods well known in the art such as optical density measurements, and the like. In a preferred embodiment, the organism is Magnaporthe grisea.

One embodiment of the invention is directed to the use of multi-well plates for screening of antibiotic compounds. The use of multi-well plates is a format that readily accommodates multiple different assays to characterize. various compounds, concentrations of compounds, and fungal organisms in varying combinations and formats. Certain testing parameters for the screening method can significantly affect the identification of growth inhibitors, and thus can be manipulated to optimize screening efficiency and/or reliability. Notable among these factors are variable sensitivities of different mutants, increasing hypersensitivity with increasingly less permissive conditions, an apparent increase in hypersensitivity with increasing compound concentration, and other factors known to those in the art.

### EXPERIMENTAL

### Example 1

### Construction of Plasmids with a Transposon Containing a Selectable Marker

Construction of Sif Transposon:

Sif was constructed using the GPS3 vector from the GPS-M mutagenesis system from New England Biolabs, Inc. (Beverly, Mass.) as a backbone. This system is based on the bacterial transposon Tn7. The following manipulations were done to GPS3 according to Sambrook et al., Molecular Cloning, a Laboratory Manual, Cold Spring Harbor Laboratory Press (1989). The kanamycin resistance gene (npt) contained between the Tn7 arms was removed by EcoRV digestion. The bacterial hygromycin B phosphotransferase (hph) gene (Gritz & Davies, 25 Gene 179 (1983) (PMID: 6319235)) under control of the Aspergillus nidulans trpC promoter and terminator (Mullaney et al., 199 Mol. Gen. Genet. 37 (1985) (PMID: 3158796)) was cloned by a HpaI/EcoRV blunt ligation into the Tn7 arms of the GPS3 vector yielding pSif1. Excision of the ampicillin resistance gene (bla) from pSif1 was achieved by cutting pSif1 with XmnI and BglI followed by a T4 DNA polymerase treatment to remove the 3′ overhangs left by the BglI digestion and religation of the plasmid to yield pSif. Top 10OF′ electrocompetent E. coli cells (Invitrogen) were transformed with ligation mixture according to manufacturer's recommendations. Transformants containing the Sif transposon were selected on LB agar (Sambrook et al., supra) containing 50 μg/ml of hygromycin B (Sigma Chem. Co., St. Louis, Mo.).

### Example 2

### Construction of a Fungal Cosmid Library

Cosmid libraries were constructed in the pcosKA5 vector (Hamer et al., 98 Proc. Nat'l. Acad. Sci. USA 5110 (2001) (PMID: 11296265)) as described in Sambrook et al. Cosmid libraries were quality checked by pulsed-field gel electrophoresis, restriction digestion analysis, and PCR identification of single genes.

### Example 3

### Construction of Cosmids with Transposon Insertion into Fungal Genes

Sif Transposition into a Cosmid:

Transposition of Sif into the cosmid framework was carried out as described by the GPS-M mutagenesis system (New England Biolabs, Inc.). Briefly, 2 μl of the 10×GPS buffer, 70 ng of supercoiled pSIF, 8-12 μg of target cosmid DNA were mixed and taken to a final volume of 20 μl with water. 1 μl of transposase (TnsABC) was added to the reaction and incubated for 10 minutes at 37° C. to allow the assembly reaction to occur. After the assembly reaction, 1 μl of start solution was added to the tube, mixed well, and incubated for 1 hour at 37° C. followed by heat inactivation of the proteins at 75° C. for 10 minutes. Destruction of the remaining untransposed pSif was completed by PISceI digestion at 37° C. for 2 hours followed by a 10 minute incubation at 75° C. to inactivate the proteins. Transformation of Top10F′ electrocompetent cells (Invitrogen) was done according to manufacturers recommendations. Sif-containing cosmid transformants were selected by growth on LB agar plates containing 50 μg/ml of hygromycin B (Sigma Chem. Co.) and 100 μg/ml of Ampicillin (Sigma Chem. Co.).

### Example 4

### High Throughput Preparation and Verification of Transposon Insertion Into the M. grisea ILV2 and ILV6 Genes

E. coli strains containing cosmids with transposon insertions were picked to 96 well growth blocks (Beckman Co.) containing 1.5 ml of TB (Terrific Broth, Sambrook et al., supra) supplemented with 50 μg/ml of ampicillin. Blocks were incubated with shaking at 37° C. overnight. E. coli cells were pelleted by centrifugation and cosmids were isolated by a modified alkaline lysis method (Marra et al., 7 Genome Res. 1072 (1997) (PMID: 9371743)). DNA quality was checked by electrophoresis on agarose gels. Cosmids were sequenced using primers from the ends of each transposon and commercial dideoxy sequencing kits (Big Dye Terminators, Perkin Elmer Co.). Sequencing reactions were analyzed on an ABI377 DNA sequencer (Perkin Elmer Co.).

The DNA sequences adjacent to the site of the transposon insertion were used to search DNA and protein databases using the BLAST algorithms (Altschul et al., supra). A single insertion of SIF into the Magnaporthe grisea ILV2 gene was chosen for further analysis. This construct was designated cpgmra0066002h11 and it contains the SIF transposon insertion within the ALS catalytic subunit-coding region. A single insertion of SIF into the Magnaporthe grisea ILV6 gene was chosen for further analysis. This construct was designated cpgmra0002003b07 and it contains the SIF transposon insertion within the ALS regulatory subunit-coding region.

### Example 5

### Preparation of ILV2 and ILV6 Cosmid DNA and Transformation of Magnaporthe grisea

Cosmid DNA from the ILV2 and ILV6 transposon tagged cosmid clones were prepared using QIAGEN Plasmid Maxi Kit (Qiagen), and digested by PI-PspI (New England Biolabs, Inc.). Fungal electro-transformation was performed essentially as described (Wu et al., 10 MPMI 700 (1997)). Briefly, M. grisea strain Guy11 was grown in complete liquid media (Talbot et al., 5 Plant Cell 1575 (1993) (PMID: 8312740)) shaking at 120 rpm for 3 days at 25° C. in the dark. Mycelia was harvested and washed with sterile H₂O and digested with 4 mg/ml beta-glucanase (InterSpex) for 4-6 hours to generate protoplasts. Protoplasts were collected by centrifugation and resuspended in 20% sucrose at a concentration of 2×10⁸ protoplasts/ml. 50 μl of protoplast suspension was mixed with 10-20 μg of the cosmid DNA and pulsed using a Gene Pulser II instrument (BioRad) set with the following parameters: 200 ohm, 25 μF, and 0.6 kV. Transformed protoplasts were regenerated in complete agar media (Talbot et al., supra) with the addition of 20% sucrose for one day, then overlayed with CM agar media containing hygromycin B (250 μg/ml) to select transformants. Transformants were screened for homologous recombination events in the target gene by PCR (Hamer et al., supra). Two independent strains were identified for ILV2 and are hereby referred to as K1-13 and K1-19, respectively. Two independent strains were identified for ILV6 and are hereby referred to as K1-6 and K1-11, respectively.

### Example 6

### Effect of Transposon Insertion on Magnaporthe Pathogenicity

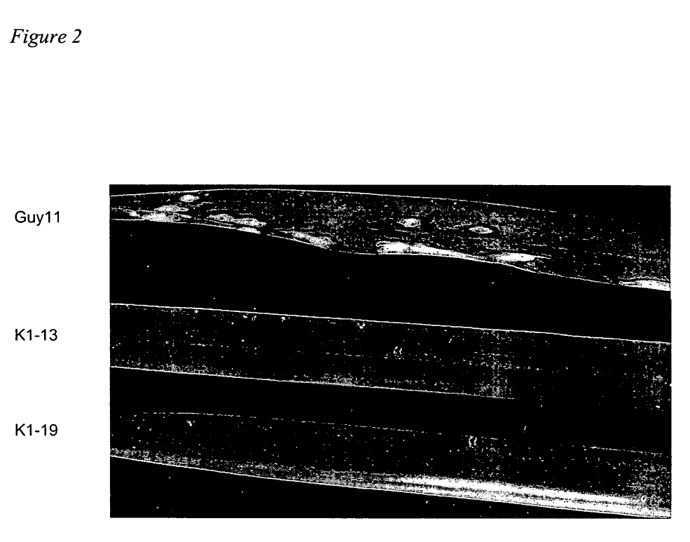

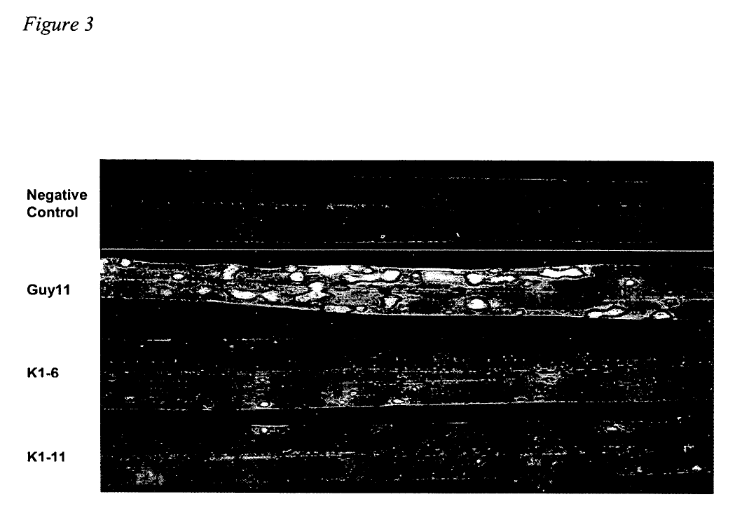

The target fungal strains for ILV2 and ILV6 obtained in Example 5 and the wild-type strain, Guy11, were subjected to a pathogenicity assay to observe infection over a 1-week period. Rice infection assays were performed using Indica rice cultivar CO39 essentially as described in Valent et al. (Valent et al., 127 Genetics 87 (1991) (PMID: 2016048)). All strains were grown for spore production on complete agar media. Spores were harvested and the concentration of spores adjusted for whole plant inoculations. Two-week-old seedlings of cultivar CO39 were sprayed with 12 ml of conidial suspension (5×10⁴ conidia per ml in 0.01% Tween-20 solution). The inoculated plants were incubated in a dew chamber at 27° C. in the dark for 36 hours, and transferred to a growth chamber (27° C. 12 hours/21° C. 12 hours at 70% humidity) for an additional 5.5 days. Leaf samples were taken at 3, 5, and 7 days post-inoculation and examined for signs of successful infection (i.e. lesions). FIGS. 2 and 3 show the effects of ILV2 and ILV6 gene disruption, respectively, on Magnaporthe infection at seven days post-inoculation.

### Example 7

### Verification of ILV6 Gene Function by Analysis of Nutritional Requirements

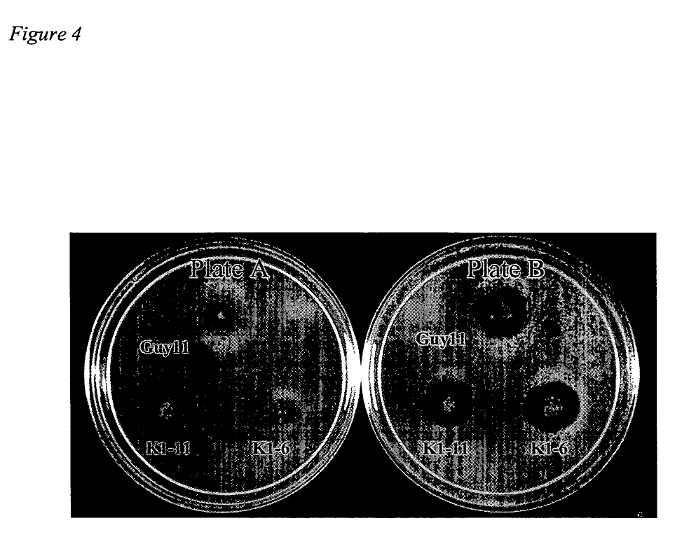

The fungal strains, K1-6 and K1-11, containing the ILV6 disrupted gene obtained in Example 5 were analyzed for their nutritional requirement for isoleucine/leucine/valine by plating each strain on minimal agar media (Talbot et al., 5 Plant Cell 1575 (1993) (PMID: 8312740)) and minimal agar media containing 4 mM each of isoleucine, leucine and valine (FIG. 4). Spores for each strain were harvested from complete media agar plates supplemented with 4 mM isoleucine, leucine and valine into 0.01% Tween 20. The spore concentrations were adjusted to 2×10⁵ spores/ml. 10 μl of spore suspension were deposited into each media. The plates were incubated at 25° C. for 7 days. Growth was assessed by comparing the growth of each mutant as compared to the wild-type strain on each media. Little growth of the mutant strains was observed on minimal media (FIG. 4, Plate A), but significant growth was observed on media containing the three amino acids (FIG. 4, Plate B) confirming the requirement for these amino acids in the ILV6 disruption mutants for wild-type growth levels.

### Example 8

### Cloning, Expression, and Isolation of ALS Catalytic and Regulatory Subunit Polypeptides

The following is a protocol to obtain isolated ALS catalytic and regulatory subunit polypeptides.

Cloning and Expression Strategies:

An ALS catalytic or regulatory subunit encoding nucleic acid is cloned into E. coli (pET vectors-Novagen), Baculovirus (Pharmingen) and Yeast (Invitrogen) expression vectors containing His/fusion protein tags, and the expression of recombinant protein is evaluated by SDS-PAGE and Western blot analysis.

Extraction:

Extract recombinant protein from 250 ml cell pellet in 3 ml of extraction buffer by sonicating 6 times, with 6 second pulses at 4° C. Centrifuge extract at 15000×g for 10 minutes and collect supernatant. Assess biological activity of the recombinant protein by activity assay.

Isolation:

Isolate recombinant protein by Ni-NTA affinity chromatography (Qiagen). Isolation protocol (perform all steps at 4° C.):
Use 3 ml Ni-beads
Equilibrate column with the buffer
Load protein extract
Wash with the equilibration buffer
Elute bound protein with 0.5M imidazole

### Example 9

### Measurement of Test Compound Binding to ALS Catalytic or Regulatory Subunit Polypeptide

The following is a protocol to identify test compounds that bind to ALS catalytic subunit polypeptide, ALS regulatory subunit polypeptide, or ALS catalytic/regulatory subunit complex.
Isolated full-length ALS catalytic subunit, ALS regulatory subunit, or ALS catalytic/regulatory subunit complex polypeptide having a His/fusion protein tag (Example 8) is bound to a HISGRAB Nickel Coated Plate (Pierce, Rockford, Ill.) following manufacturer's instructions.
Buffer conditions are optimized (e.g. ionic strength or pH, Shoolingin-Jordan et al., 281 Methods Enzymol: 309-16 (1997) (PMID: 9250995)) for binding of radiolabeled pyruvate or 2-acetolactate to the bound ALS polypeptide.
Screening of test compounds is performed by adding test compound and radioactively labeled pyruvate or 2-acetolactate to the wells of the HISGRAB plate containing bound ALS polypeptide.
The wells are washed to remove excess labeled ligand and scintillation fluid (SCINTIVERSE, Fisher Scientific) is added to each well.
The plates are read in a microplate scintillation counter.
Candidate compounds are identified as wells with lower radioactivity as compared to control wells with no test compound added.

Additionally, isolated polypeptides comprising 10-50 amino acids of M. grisea ALS catalytic or regulatory subunit polypeptides are screened in the same way. A polypeptide comprising 10-50 amino acids is generated by subcloning a portion of the ALS catalytic or regulatory subunit encoding nucleic acid into a protein expression vector that adds a His-Tag when expressed (see Example 8). Oligonucleotide primers are designed to amplify a portion of the ALS catalytic or regulatory subunit coding region using the polymerase chain reaction amplification method. The DNA fragment encoding a polypeptide of 10-50 amino acids is cloned into an expression vector, expressed in a host organism and isolated as described in Example 8 above.

Test compounds that bind ALS catalytic subunit, ALS regulatory subunit, or ALS catalytic/regulatory subunit complex polypeptide are further tested for antibiotic activity. M. grisea is grown as described for spore production on oatmeal agar media (Talbot et al., supra). Spores are harvested into minimal media to a concentration of 2×10⁵ spores/ml and the culture is divided. Id. The test compound is added to one culture to a final concentration of 20-100 μg/ml. Solvent only is added to the second culture. The growth of the solvent containing culture and the test compound containing culture are compared. A test compound is an antibiotic candidate if the growth of the culture containing the test compound is less than the growth of the control culture.

Test compounds that bind ALS catalytic subunit, ALS regulatory subunit, or ALS catalytic/regulatory subunit complex polypeptide are further tested for antipathogenic activity. M. grisea is grown as described for spore production on oatmeal agar media (Talbot et al., supra). Spores are harvested into water with 0.01% Tween 20 to a concentration of 5×10⁴ spores/ml and the spore suspension is divided. Id. The test compound is added to one spore suspension to a final concentration of 20-100 μg/ml. Solvent only is added to the second spore suspension. Rice infection assays are performed using Indica rice cultivar CO39 essentially as described in Valent et al., supra). Two-week-old seedlings of cultivar CO39 are sprayed with 12 ml of conidial suspension. The inoculated plants are incubated in a dew chamber at 27° C. in the dark for 36 hours, and transferred to a growth chamber (27° C. 12 hours/21° C. 12 hours at 70% humidity) for an additional 5.5 days. Leaf samples are examined at 5 days post-inoculation to determine the extent of pathogenicity as compared to the control samples.

Alternatively, antipathogenic activity can be assessed using an excised leaf pathogenicity assay. Spore suspensions are prepared in water only to a concentration of 5×10⁴ spores/ml and the culture is divided. The test compound is added to one culture to a final concentration of 20-100 μg/ml. Solvent only is added to the second culture. Detached leaf assays are performed by excising lcm segments of rice leaves from Indica rice cultivar CO39 and placing them on 1% agarose in water. 10 μl of each spore suspension is place on the leaf segments and the samples are incubated at 25° C. for 5 days in the dark. Leaf samples are examined at 5 days post-inoculation to determine the extent of pathogenicity as compared to the control samples.

### Example 10

### Assays for Identification of Inhibitors of ALS Enzymatic Activity

The ability of a compound to inhibit ALS catalytic subunit activity is detected using in vitro enzymatic assays in which the disappearance of a substrate or the appearance of a product is directly or indirectly detected. The ability of a test compound to specifically inhibit activity of ALS regulatory subunit is measured by monitoring the effect of the presence of the compound on the progression of ALS catalytic subunit enzymatic activity in the presence and absence of the regulatory subunit. Suitable methods and reaction conditions and buffers for measuring ALS enzymatic activity are, for example, as described in Pang, S. S. and Duggleby R. G. (1999), supra, herein incorporated by reference in its entirety.

An exemplary assay for identifying compounds that inhibit ALS catalytic subunit activity is as follows:
1. Contact an ALS catalytic subunit polypeptide with a potassium phosphate buffer reaction mixture comprising 50 mM pyruvate, 1 mM thiamin diphosphate, 10 mM MgCl₂, and 10 μM flavin adenine dinucleotide at pH 7.0 and 30° C. for 20 minutes in the presence and absence of a test compound.
2. Add a sufficient amount of a 50% H₂SO₄ solution to the reaction mixtures to give a final concentration of 1%.
3. Incubate the reaction mixtures at 60° C. for 15 minutes (2-acetolactate is converted to acetoin).
4. Quantify acetoin by adding creatine and α-naphthol to the reaction mixtures to a concentration of 0.15% creatine and 1.54% α-naphthol, incubating the reactions at 60° C. for 15 minutes, and measuring the absorbance of acetoin in the reaction mixtures at 525 nM.
5. Compare the concentration of 2-acetolactate generated in the reactions in the presence and absence of the test compound (a decrease in the amount of 2-acetolactate produced in the presence, relative to the absence, of the compound indicates that the test compound is a candidate for an antibiotic.

Another assay to identify compounds that inhibit ALS enzymatic activity is a modification of the assay described above. In this case, the assay is performed in the same manner as described above with the exception of using an ALS catalytic/regulatory subunit complex in place of the ALS catalytic subunit in step (1). Compounds are similary identified as candidate antibiotics by measuring a decrease in the amount of 2-acetolactate produced in the presence, relative to the absence, of the compound.

Candidate antibiotic compounds are additionally determined in either manner using a polypeptide comprising a fragment of the M. grisea ALS catalytic subunit. The ALS catalytic subunit fragment is generated by subcloning a portion of the ALS catalytic subunit encoding nucleic acid into a protein expression vector that adds a His-Tag when expressed (see Example 8). Oligonucleotide primers are designed to amplify a portion of the ALS catalytic subunit-coding region using polymerase chain reaction amplification method. The DNA fragment encoding the ALS catalytic subunit polypeptide fragment is cloned into an expression vector, expressed and isolated as described in Example 8 above.

Test compounds identified as inhibitors of ALS catalytic subunit activity are further tested for antibiotic activity. Magnaporthe grisea fungal cells are grown under standard fungal growth conditions that are well known and described in the art. M. grisea is grown as described for spore production on oatmeal agar media (Talbot et al., supra). Spores are harvested into minimal media to a concentration of 2×10⁵ spores/ml and the culture is divided. Id. The test compound is added to one culture to a final concentration of 20-100 μg/ml. Solvent only is added to the second culture. The growth of the solvent containing culture and the test compound containing culture are compared. A test compound is an antibiotic candidate if the growth of the culture containing the test compound is less than the growth of the control culture.

Test compounds identified as inhibitors of ALS catalytic subunit activity are further tested for antipathogenic activity. M. grisea is grown as described for spore production on oatmeal agar media (Talbot et al., supra). Spores are harvested into water with 0.01% Tween 20 to a concentration of 5×10⁴ spores/ml and the culture is divided. Id. The test compound is added to one culture to a final concentration of 20-100 μg/ml. Solvent only is added to the second culture. Rice infection assays are performed using Indica rice cultivar CO39 essentially as described in Valent et al., supra. Two-week-old seedlings of cultivar CO39 are sprayed with 12 ml of conidial suspension. The inoculated plants are incubated in a dew chamber at 27° C. in the dark for 36 hours, and transferred to a growth chamber (27° C. 12 hours/21° C. 12 hoursat 70% humidity) for an additional 5.5 days. Leaf samples are examined at 5-7 days post-inoculation to determine the extent of pathogenicity as compared to the control samples.

Alternatively, antipathogenic activity is assessed using an excised leaf pathogenicity assay. Spore suspensions are prepared in water only to a concentration of 5×10⁴ spores/ml and the culture is divided. The test compound is added to one culture to a final concentration of 20-100 μg/ml. Solvent only is added to the second culture. Detached leaf assays are performed by excising lcm segments of rice leaves from Indica rice cultivar CO39 and placing them on 1% agarose in water. 10 μl of each spore suspension is place on the leaf segments and the samples are incubated at 25° C. for 5 days in the dark. Leaf samples are examined at 5 days post-inoculation to determine the extent of pathogenicity as compared to the control samples.

### Example 11

### Assays for Indentification of Inhibitors of ALS Regulatory Subunit Activity

A compound is identified as an inhibitor of ALS regulatory subunit activity by measuring a decrease in activity of ALS catalytic/regulatory subunit complex in the presence, relative to the absence, of the test compound and measuring no effect of the test compound on activity of ALS catalytic subunit alone. An assay for identifying compounds that specifically inhibit ALS regulatory subunit function is similar to that described above in Example 10 with the addition of ALS regulatory subunit. The assay is as follows:
1. Contact an ALS catalytic and regulatory subunit polypeptide complex with a potassium phosphate buffer reaction mixture comprising 50 mM pyruvate, 1 mM thiamin diphosphate, 10 mM MgCl₂, and 10 μM flavin adenine dinucleotide at pH 7.0 and 30° C. for 20 minutes in the presence and absence of a test compound.
2. Contact the ALS catalytic subunit polypeptide alone with the same reaction mixture at 30° C. for 20 minutes in the presence and absence of the test compound.
3. Add a sufficient amount of a 50% H₂SO₄ solution to each of the reaction mixtures of steps (1) and (2) to give a final concentration of 1%.
4. Incubate each of the reaction mixtures of step (3) at 60° C. for 15 minutes (2-acetolactate is converted to acetoin).
5. Quantify acetoin by adding creatine and α-naphthol to each of the reaction mixtures of step (4) to a concentration of 0.15% creatine and 1.54% α-naphthol, incubating each of the reaction mixtures at 60° C. for 15 minutes, and measuring the absorbance of acetoin in each of the reaction mixtures at 525 nM.
6. Compare the concentration of 2-acetolactate generated in the reactions in the presence and absence of the test compound in steps (1) and (2). A decrease in the amount of 2-acetolactate produced in the presence, relative to the absence, of the compound in step (1) and no change in 2-acetolactate in step (2) indicates that the test compound is a candidate for an antibiotic.

Candidate antibiotic compounds are additionally determined in the same manner using a polypeptide comprising a fragment of the M. grisea ALS regulatory subunit. The ALS regulatory subunit fragment is generated by subcloning a portion of the ALS regulatory subunit encoding nucleic acid into a protein expression vector that adds a His-Tag when expressed (see Example 8). Oligonucleotide primers are designed to amplify a portion of the ALS regulatory subunit coding region using polymerase chain reaction amplification method. The DNA fragment encoding the ALS regulatory subunit polypeptide fragment is cloned into an expression vector, expressed and isolated as described in Example 8 above.

Test compounds identified as inhibitors of ALS regulatory subunit activity are further tested for antibiotic activity by measuring the effect of the test compound on Magnaporthe grisea fungal growth and pathogenicity as described above in Example 10.

### Example 12

### Assays for Testing Compounds for Alteration of ALS Catalytic and Regulatory Subunit Gene Expression

Magnaporthe grisea fungal cells are grown under standard fungal growth conditions that are well known and described in the art. Wild-type M. grisea spores are harvested from cultures grown on complete agar or oatmeal agar media after growth for 10-13 days in the light at 25° C. using a moistened cotton swab. The concentration of spores is determined using a hemacytometer and spore suspensions are prepared in a minimal growth medium to a concentration of 2×10⁵ spores per ml. 25 ml cultures are prepared to which test compounds will be added at various concentrations. A culture with no test compound present is included as a control. The cultures are incubated at 25° C. for 3 days after which test compound or solvent only control is added. The cultures are incubated an additional 18 hours. Fungal mycelia is harvested by filtration through Miracloth (CalBiochem, La Jolla, Calif.), washed with water, and frozen in liquid nitrogen. Total RNA is extracted with TRIZOL Reagent using the methods provided by the manufacturer (Life Technologies, Rockville, Md.). Expression is analyzed by Northern analysis of the RNA samples as described (Sambrook et al., supra) using a radiolabeled fragment of the ALS catalytic or regulatory subunit encoding nucleic acid as a probe. Test compounds resulting in an altered level of ALS catalytic or regulatory subunit mRNA relative to the untreated control sample are identified as candidate antibiotic compounds.

Test compounds identified as inhibitors of ALS catalytic or regulatory subunit expression are further tested for antibiotic activity by measuring the effect of the test compound on Magnaporthe grisea fungal growth and pathogenicity as described above in Example 10.

### Example 13

### In Vivo Cell Based Assay Screening Protocol with a Fungal Strain Containing a Mutant Form of ALS Catalytic or Regulatory Subunit that Lacks Activity

The effect of test compounds on the growth of wild-type fungal cells and mutant fungal cells having a mutant ALS catalytic or regulatory subunit gene is measured and compared as follows. Magnaporthe grisea fungal cells containing a mutant form of the ALS catalytic or regulatory subunit gene that lacks activity, for example an ALS catalytic or regulatory subunit gene containing a transposon insertion, are grown under standard fungal growth conditions that are well known and described in the art. Magnaporthe grisea spores are harvested from cultures grown on complete agar medium containing L-branched chain amino acids, leucine, valine, isoleucine, (Sigma) after growth for 10-13 days in the light at 25° C. using a moistened cotton swab. The concentration of spores is determined using a hemacytometer and spore suspensions are prepared in a minimal growth medium containing L-branched chain amino acids to a concentration of 2×10⁵ spores per ml. Approximately 4×10⁴ spores are added to each well of 96-well plates to which a test compound is added (at varying concentrations). The total volume in each well is 200 μl. Wells with no test compound present (growth control), and wells without cells are included as controls (negative control). The plates are incubated at 25° C. for seven days and optical density measurements at 590 nm are taken daily. Wild-type cells are screened under the same conditions.

The effect of each of the test compounds on the mutant and wild-type fungal cells is measured against the growth control and the percent of inhibition is calculated as the OD₅₉₀ (fungal strain plus test compound)/OD₅₉₀ (growth control)×100. The percent of growth inhibition in the presence of the test compound on the mutant and wild-type fungal strains are compared. Compounds that show differential growth inhibition between the mutant and the wild-type cells are identified as potential antifungal compounds. Similar protocols may be found in Kirsch & DiDomenico, 26 Biotechnology 177 (1994) (PMID: 7749303)). Test compounds that produce a differential growth response between the mutant and wild type fungal strains are further tested for antipathogenic activity as described above in Example 10.

### Example 14

### In Vivo Cell Based Assay Screening Protocol with a Fungal Strain Containing a Mutant Form of ALS Catalytic or Regulatory Subunit with Reduced Activity

The effect of test compounds on the growth of wild-type fungal cells and mutant fungal cells having a mutant ALS catalytic or regulatory subunit gene is measured and compared as follows. Magnaporthe grisea fungal cells containing a mutant form of the ALS catalytic or regulatory subunit gene resulting in reduced activity, such as a transposon insertion mutation in a regulatory region of the gene or a promoter truncation mutation that reduces expression, are grown under standard fungal growth conditions that are well known and described in the art. A promoter truncation is made by deleting a portion of the promoter upstream of the transcription start site using standard molecular biology techniques that are well known and described in the art (Sambrook et al., supra).

The mutant and wild-type Magnaporthe grisea spores are harvested from cultures grown on complete agar medium containing L-branched chain amino acids (Sigma) after growth for 10-13 days in the light at 25° C. using a moistened cotton swab. The concentration of spores is determined using a hemacytometer and spore suspensions are prepared in a minimal growth medium to a concentration of 2×10⁵ spores per ml. Approximately 4×10⁴ spores are added to each well of 96-well plates to which a test compound is added (at varying concentrations). The total volume in each well is 200 μl. Wells with no test compound present (growth control), and wells without cells are included as controls (negative control). The plates are incubated at 25° C. for seven days and optical density measurements at 590 nm are taken daily. Wild-type cells are screened under the same conditions.

The effect of each test compound on the mutant and wild-type fungal strains is measured against the growth control and the percent of inhibition is calculated as the OD₅₉₀ (fungal strain plus test compound)/OD₅₉₀ (growth control)×100. The percent growth inhibition as a result of each of the test compounds on the mutant and wild-type cells is compared. Compounds that show differential growth inhibition between the mutant and the wild-type cells are identified as potential antifungal compounds. Similar protocols may be found in Kirsch & DiDomenico, supra. Test compounds that produce a differential growth response between the mutant and wild type fungal strains are further tested for antipathogenic activity as described above in Example 10.

### Example 15

### In Vivo Cell Based Assay Screening Protocol with a Fungal Strain Containing a Mutant Form of a Branched Chain Amino Acid Biosynthetic Gene that Lacks Activity

The effect of test compounds on the growth of wild-type fungal cells and mutant fungal cells having a mutant form of a gene in the branched chain amino acid biosynthetic pathway is measured and compared as follows. Magnaporthe grisea fungal cells containing a mutant form of a gene that lacks activity in the branched chain amino acid biosynthetic pathway (e.g. ketol-acid reductoisomerase or dihydroxy-acid dehydratase having a transposon insertion) are grown under standard fungal growth conditions that are well known and described in the art. Magnaporthe grisea spores are harvested from cultures grown on complete agar medium containing L-branched chain amino acids, leucine, valine, isoleucine, (Sigma) after growth for 10-13 days in the light at 25° C. using a moistened cotton swab. The concentration of spores is determined using a hemacytometer and spore suspensions are prepared in a minimal growth medium containing L-branched chain amino acids (4 mM) to a concentration of 2×10⁵ spores per ml.

Approximately 4×10⁴ spores or cells are harvested and added to each well of 96-well plates to which growth media is added in addition to an amount of test compound (at varying concentrations). The total volume in each well is 200 μl. Wells with no test compound present, and wells without cells are included as controls. The plates are incubated at 25° C. for seven days and optical density measurements at 590 nm are taken daily. Wild-type cells are screened under the same conditions.

The effect of each compound on the mutant and wild-type fungal strains is measured against the growth control and the percent of inhibition is calculated as the OD₅₉₀ (fungal strain plus test compound)/OD₅₉₀ (growth control)×100. The percent of growth inhibition as a result of each of the test compounds on the mutant and the wild-type cells are compared. Compounds that show differential growth inhibition between the mutant and the wild-type cells are identified as potential antifungal compounds. Similar protocols may be found in Kirsch & DiDomenico, supra. Test compounds that produce a differential growth response between the mutant and wild type fingal strains are further tested for antipathogenic activity as described above in Example 10.

### Example 16

### In Vivo Cell Based Assay Screening Protocol with a Fungal Strain Containing a Mutant Form of a Branched Chain Amino Acid Biosynthetic Gene with Reduced Activity

The effect of test compounds on the growth of wild-type fungal cells and mutant fungal cells having a mutant form of a gene in the branched chain amino acid biosynthetic pathway is measured and compared as follows. Magnaporthe grisea fungal cells containing a mutant form of a gene resulting in reduced protein activity in the branched chain amino acid biosynthetic pathway (e.g. ketol-acid reductoisomerase or dihydroxy-acid dehydratase having a promoter truncation that reduces expression), are grown under standard fungal growth conditions that are well known and described in the art. Mutant and wild-type Magnaporthe grisea spores are harvested from cultures grown on complete agar medium containing L-branched chain amino acids (Sigma) after growth for 10-13 days in the light at 25° C. using a moistened cotton swab. The concentration of spores is determined using a hemacytometer and spore suspensions are prepared in a minimal growth medium to a concentration of 2×10⁵ spores per ml.

Approximately 4×10⁴ spores or cells are harvested and added to each well of 96-well plates to which growth media is added in addition to an amount of test compound (at varying concentrations). The total volume in each well is 200 μl. Wells with no test compound present, and wells without cells are included as controls. The plates are incubated at 25° C. for 7 days and optical density measurements at 590 nm are taken daily. Wild-type cells are screened under the same conditions. The effect of each compound on the mutant and wild-type fungal strains is measured against the growth control and the percent of inhibition is calculated as the OD₅₉₀ (fungal strain plus test compound)/OD₅₉₀ (growth control)×100. The percent of growth inhibition as a result of each of the test compounds on the mutant and wild-type cells are compared. Compounds that show differential growth inhibition between the mutant and the wild-type cells are identified as potential antifungal compounds. Similar protocols may be found in Kirsch & DiDomenico, supra. Test compounds that produce a differential growth response between the mutant and wild type fungal strains are further tested for antipathogenic activity as described above in Example 10.

### Example 17

### In Vivo Cell Based Assay Screening Protocol with a Fungal Strain Containing a Heterologous ALS Catalytic or Regulatory Subunit Gene

The effect of test compounds on the growth of wild type fungal cells and fungal cells lacking a functional endogenous ALS catalytic or regulatory subunit encoding gene and containing a heterologous ALS catalytic or regulatory subunit encoding gene is measured and compared as follows. Wild type M. grisea fungal cells and M. grisea fungal cells lacking an endogenous ALS catalytic or regulatory subunit encoding gene and containing a heterologous ALS catalytic or regulatory subunit encoding gene from Neurospora crassa (Genbank Accession No. CAB91255), are grown under standard fungal growth conditions that are well known and described in the art.

A M. grisea strain carrying a heterologous ALS catalytic or regulatory subunit gene is made as follows. A M. grisea strain is made with a nonfunctional endogenous ALS catalytic or regulatory subunit gene, such as one containing a transposon insertion in the native gene that abolishes protein activity. A construct containing a heterologous ALS catalytic or regulatory subunit gene is made by cloning a heterologous ALS catalytic or regulatory subunit gene, such as from Neurospora crassa, into a fungal expression vector containing a trpC promoter and terminator (e.g. Carroll et al., 41 Fungal Gen. News Lett. 22 (1994) (describing pCB1003) using standard molecular biology techniques that are well known and described in the art (Sambrook et al., supra). The vector construct is used to transform the M. grisea strain lacking a functional endogenous ALS catalytic or regulatory subunit gene. Fungal transformants containing a functional ALS catalytic or regulatory subunit gene are selected on minimal agar medium lacking L-branched chain amino acids, as only transformants carrying a functional ALS catalytic or regulatory subunit gene grow in the absence of L-branched chain amino acids.

Wild-type strains of M. grisea and strains containing a heterologous form of ALS catalytic or regulatory subunit are grown under standard fungal growth conditions that are well known and described in the art. M. grisea spores are harvested from cultures grown on complete agar medium after growth for 10-13 days in the light at 25° C. using a moistened cotton swab. The concentration of spores is determined using a hemacytometer and spore suspensions are prepared in a minimal growth medium to a concentration of 2×10⁵ spores per ml.

Approximately 4×10⁴ spores or cells are harvested and added to each well of 96-well plates to which growth media is added in addition to an amount of test compound (at varying concentrations). The total volume in each well is 200 μl. Wells with no test compound present, and wells without cells are included as controls. The plates are incubated at 25° C. for seven days and optical density measurements at 590 nm are taken daily. The effect of each compound on the wild type and heterologous fungal strains is measured against the growth control and the percent of inhibition is calculated as the OD₅₉₀ (fungal strain plus test compound)/OD₅₉₀ (growth control)×100. The percent of growth inhibition as a result of each of the test compounds on the wild type and heterologous fungal strains are compared. Compounds that show differential growth inhibition between the wild type and heterologous strains are identified as potential antifungal compounds with specificity to the native or heterologous ALS catalytic or regulatory subunit gene products. Similar protocols may be found in Kirsch & DiDomenico, supra. Test compounds that produce a differential growth response between the strain containing a heterologous gene and strain containing a fungal gene are further tested for antipathogenic activity as described above in Example 10.

### Example 18

### Pathway Specific In Vivo Assay Screening Protocol

Compounds are tested as candidate antibiotics as follows. Magnaporthe grisea fungal cells are grown under standard fungal growth conditions that are well known and described in the art. Wild-type M. grisea spores are harvested from cultures grown on oatmeal agar media after growth for 10-13 days in the light at 25° C. using a moistened cotton swab. The concentration of spores is determined using a hemocytometer and spore suspensions are prepared in a minimal growth medium and a minimal growth medium containing L-branched chain amino acids (Sigma) to a concentration of 2×10⁵ spores per ml. The minimal growth media contains carbon, nitrogen, phosphate, and sulfate sources, and magnesium, calcium, and trace elements (for example, see innoculating fluid in Example 7). Spore suspensions are added to each well of a 96-well microtiter plate (approximately 4×10⁴ spores/well). For each well containing a spore suspension in minimal media, an additional well is present containing a spore suspension in minimal medium containing L-branched chain amino acids.

Test compounds are added to wells containing spores in minimal media and minimal media containing L-branched chain amino acids. The total volume in each well is 200 μl. Both minimal media and L-branched chain amino acid containing media wells with no test compound are provided as controls. The plates are incubated at 25° C. for seven days and optical density measurements at 590 nm are taken daily. A compound is identified as a candidate for an antibiotic acting against the L-branched chain amino acid biosynthetic pathway when the observed growth in the well containing minimal media is less than the observed growth in the well containing L-branched chain amino acids as a result of the addition of the test compound. Similar protocols may be found in Kirsch & DiDomenico, supra.

Published references and patent publications cited herein are incorporated by reference as if terms incorporating the same were provided upon each occurrence of the individual reference or patent document. While the foregoing describes certain embodiments of the invention, it will be understood by those skilled in the art that variations and modifications may be made that will fall within the scope of the invention. The foregoing examples are intended to exemplify various specific embodiments of the invention and do not limit its scope in any manner.

1. A method for identifying a test compound as a candidate for an antibiotic, comprising:
a) contacting an ALS polypeptide with a test compound, wherein the ALS polypeptide is selected from the group consisting of:
i) an ALS catalytic subunit polypeptide;
ii) an ALS regulatory subunit polypeptide; and
iii) an ALS catalytic subunit polypeptide and an ALS regulatory subunit polypeptide; and
b) detecting the presence or absence of binding between the test compound and the ALS polypeptide, wherein binding indicates that the test compound is a candidate for an antibiotic.

2. The method of claim 1, wherein the ALS polypeptide is a fungal ALS polypeptide.

3. The method of claim 1, wherein the ALS polypeptide is a Magnaporthe ALS polypeptide.

4. The method of claim 1, wherein the ALS polypeptide is SEQ ID NO:2.

5. The method of claim 1, wherein the ALS polypeptide is SEQ ID NO:5.

6. The method of claim 1, wherein the the ALS polypeptide is SEQ ID NO:2 and SEQ ID NO:5

7. The method of claim 1, wherein the the ALS polypeptide is selected from the group consisting of:
a) an ALS polypeptide consisting essentially of SEQ ID NO:2;
b) an ALS polypeptide consisting essentially of SEQ ID NO:5;
c) an ALS polypeptide consisting essentially of SEQ ID NO:2 and an ALS polypeptide consisting essentially of SEQ ID NO:5;
d) an ALS polypeptide having at least ten consecutive amino acids of SEQ ID NO:2;
e) an ALS polypeptide having at least ten consecutive amino acids of SEQ ID NO:5;
f) an ALS polypeptide having at least ten consecutive amino acids of SEQ ID NO:2 and an ALS polypeptide having at least ten consecutive amino acids of SEQ ID NO:5;
g) an ALS polypeptide having at least 50% sequence identity with SEQ ID NO:2 and at least 10% of the activity of SEQ ID NO:2;
h) an ALS polypeptide having at least 50% sequence identity with SEQ ID NO:5 and at least 10% of the activity of SEQ ID NO:5;
i) an ALS polypeptide having at least 50% sequence identity with SEQ ID NO:2 and at least 10% of the activity of SEQ ID NO:2 and an ALS polypeptide having at least 50% sequence identity with SEQ ID NO:5 and at least 10% of the activity of SEQ ID NO:5;
j) an ALS polypeptide consisting of at least 50 amino acids having at least 50% sequence identity with SEQ ID NO:2;
k) an ALS polypeptide consisting of at least 50 amino acids having at least 50% sequence identity with SEQ ID NO:5; and
l) an ALS polypeptide consisting of at least 50 amino acids having at least 50% sequence identity with SEQ ID NO:2 and an ALS polypeptide consisting of at least 50 amino acids having at least 50% sequence identity with SEQ ID NO:5.

8. A method for identifying a test compound as a candidate for an antibiotic, comprising:
a) contacting an ALS catalytic subunit polypeptide with a reaction mixture comprising pyruvate, in the presence and absence of a test compound;
b) contacting the ALS catalytic subunit polypeptide and an ALS regulatory subunit polypeptide with the reaction mixture comprising pyruvate, in the presence and absence of the test compound; and
c) comparing the concentration of one or more of pyruvate, 2-acetolactate and/or CO₂ in steps (a) and (b), wherein no change in concentration in step (a) versus a change in concentration in step (b), in the presence, relative to the absence, of the test compound, indicates that the test compound is a candidate for an antibiotic.

9. The method of claim 8, wherein the ALS catalytic subunit polypeptide and the ALS regulatory subunit polypeptide are fungal ALS polypeptides.

10. The method of claim 9, wherein the ALS catalytic subunit polypeptide and the ALS regulatory subunit polypeptide are Magnaporthe ALS polypeptides.

11. The method of claim 8, wherein the ALS catalytic subunit polypeptide is SEQ ID NO:2 and the ALS regulatory subunit polypeptide is SEQ ID NO:5

12. The method of claim 8, wherein the ALS catalytic subunit polypeptide is selected from the group consisting of:
a) a polypeptide consisting essentially of SEQ ID NO:2;
a) a polypeptide having at least 50% sequence identity with SEQ ID NO:2 and at least 10% of the activity of SEQ ID NO:2;
b) a polypeptide comprising at least 50 consecutive amino acids of SEQ ID NO:2 and having at least 10% of the activity of SEQ ID NO:2; and
d) a polypeptide comprising at least 50 amino acids having at least 50% sequence identity with SEQ ID NO:2 and having at least 10% of the activity of SEQ ID NO:2.

13. The method of claim 8, wherein the ALS regulatory subunit polypeptide is selected from the group consisting of:
a) a polypeptide consisting essentially of SEQ ID NO:5;
b) a polypeptide having at least 50% sequence identity with SEQ ID NO:5 and at least 10% of the activity of SEQ ID NO:5;
c) a polypeptide comprising at least 50 consecutive amino acids of SEQ ID NO:5 and having at least 10% of the activity of SEQ ID NO:5; and
d) a polypeptide comprising at least 50 amino acids having at least 50% sequence identity with SEQ ID NO:5 and having at least 10% of the activity of SEQ ID NO:5.

14. A method for identifying a test compound as a candidate for an antibiotic, comprising:
a) contacting an ALS catalytic subunit polypeptide with a reaction mixture comprising pyruvate, in the presence and absence of a test compound; and
b) comparing the concentration of one or more of pyruvate, 2-acetolactate and/or CO₂ in step (a), wherein a change in concentration in the presence, relative to the absence, of the test compound indicates that the test compound is a candidate for an antibiotic.

15. The method of claim 14, wherein the ALS catalytic subunit polypeptide is a fungal polypeptide.

16. The method of claim 14, wherein the ALS catalytic subunit polypeptide is a Magnaporthe polypeptide.

17. The method of claim 14, wherein the ALS catalytic subunit polypeptide is SEQ ID NO:2

18. The method of claim 14, wherein the ALS catalytic subunit polypeptide is selected from the group consisting of:
a) a polypeptide consisting essentially of SEQ ID NO:2;
b) a polypeptide having at least 50% sequence identity with SEQ ID NO:2 and at least 10% of the activity of SEQ ID NO:2;
c) a polypeptide comprising at least 50 consecutive amino acids of SEQ ID NO:2 and having at least 10% of the activity of SEQ ID NO:2; and
d) a polypeptide comprising at least 50 amino acids having at least 50% sequence identity with SEQ ID NO:2 and having at least 10% of the activity of SEQ ID NO:2.

19. A method for identifying a test compound as a candidate for an antibiotic, comprising:
a) contacting an ALS catalytic subunit polypeptide and an ALS regulatory subunit polypeptide with a reaction mixture comprising pyruvate, in the presence and absence of a test compound; and
b) comparing the concentration of one or more of pyruvate, 2-acetolactate and/or CO₂ in step (a), wherein a change in concentration in the presence, relative to the absence, of the test compound indicates that the test compound is a candidate for an antibiotic.

20. The method of claim 19, wherein the ALS catalytic subunit polypeptide and the ALS regulatory subunit polypeptide are fungal polypeptides.

21. The method of claim 19, wherein the ALS catalytic subunit polypeptide and the ALS regulatory subunit polypeptide are Magnaporthe polypeptides.

22. The method of claim 19, wherein the ALS catalytic subunit polypeptide is SEQ ID NO:2 and the ALS regulatory subunit polypeptide is SEQ ID NO:5

23. The method of claim 19, wherein the ALS catalytic subunit polypeptide is selected from the group consisting of:
a) a polypeptide consisting essentially of SEQ ID NO:2;
b) a polypeptide having at least 50% sequence identity with SEQ ID NO:2 and at least 10% of the activity of SEQ ID NO:2;
c) a polypeptide comprising at least 50 consecutive amino acids of SEQ ID NO:2 and having at least 10% of the activity of SEQ ID NO:2; and
d) a polypeptide comprising at least 50 amino acids having at least 50% sequence identity with SEQ ID NO:2 and having at least 10% of the activity of SEQ ID NO:2.

24. The method of claim 19, wherein the ALS regulatory subunit polypeptide is selected from the group consisting of:
a) a polypeptide consisting essentially of SEQ ID NO:5;
b) a polypeptide having at least 50% sequence identity with SEQ ID NO:5 and at least 10% of the activity of SEQ ID NO:5;
c) a polypeptide comprising at least 50 consecutive amino acids of SEQ ID NO:5 and having at least 10% of the activity of SEQ ID NO:5; and
d) a polypeptide comprising at least 50 amino acids having at least 50% sequence identity with SEQ ID NO:5 and having at least 10% of the activity of SEQ ID NO:5.

25. A method for identifying a test compound as a candidate for an antibiotic, comprising:
a) measuring the expression of an ALS catalytic and/or regulatory subunit in an organism, or a cell or tissue thereof, in the presence and absence of a test compound; and
b) comparing the expression of the ALS catalytic and/or regulatory subunit in the presence and absence of the test compound, wherein an altered expression in the presence of the test compound indicates that the test compound is a candidate for an antibiotic.

26. The method of claim 25, wherein the organism is a fungus.

27. The method of claim 25, wherein the organism is Magnaporthe.

28. The method of claim 25, wherein the ALS catalytic subunit is SEQ ID NO:2.

29. The method of claim 25, wherein the ALS regulatory subunit is SEQ ID NO:5.

30. The method of claim 25, wherein the expression of the ALS catalytic and/or regulatory subunit is measured by detecting the ALS catalytic and/or regulatory subunit mRNA.

31. The method of claim 25, wherein the expression of the ALS catalytic and/or regulatory subunit is measured by detecting the ALS catalytic and/or regulatory subunit polypeptide.

32. The method of claim 25, wherein the expression of the ALS catalytic and/or regulatory subunit is measured by detecting the ALS catalytic and/or regulatory subunit polypeptide activity.

33. A method for identifying a test compound as a candidate for an antibiotic, comprising:
a) providing a fungal organism having a first form of an ALS catalytic subunit;
b) providing a fungal organism having a second form of the ALS catalytic subunit, wherein one of the first or the second form of the ALS catalytic subunit has at least 10% of the activity of SEQ ID NO:2; and
c) determining the growth of the organism having the first form of the ALS catalytic subunit and the organism having the second form of the ALS catalytic subunit in the presence of a test compound,
wherein a difference in growth between the two organisms in the presence of the test compound indicates that the test compound is a candidate for an antibiotic.

34. The method of claim 33, wherein the fungal organism having the first form of the ALS catalytic subunit and the fungal organism having the second form of the ALS catalytic subunit are Magnaporthe and the first and the second form of the ALS catalytic subunit are fungal ALS catalytic subunits.

35. The method of claim 33, wherein the first form of the ALS catalytic subunit is SEQ ID NO:1.

36. The method of claim 33, wherein the fungal organism having the first form of the ALS catalytic subunit and the fungal organism having the second form of the ALS catalytic subunit are Magnaporthe and the first form of the ALS catalytic subunit is SEQ ID NO:1.

37. The method of claim 33, wherein the fungal organism having the first form of the ALS catalytic subunit and the fungal organism having the second form of the ALS catalytic subunit are Magnaporthe, the first form of the ALS catalytic subunit is SEQ ID NO:1, and the second form of the ALS catalytic subunit is a heterologous ALS catalytic subunit.

38. The method of claim 33, wherein the fungal organism having the first form of the ALS catalytic subunit and the fungal organism having the second form of the ALS catalytic subunit are Magnaporthe, the first form of the ALS catalytic subunit is SEQ ID NO:1, and the second form of the ALS catalytic subunit is SEQ ID NO:1 comprising a transposon insertion that reduces or abolishes ALS catalytic subunit activity.

39. A method iror identifying a test compound as a candidate for an antibiotic, comprising:
a) providing a fungal organism having a first form of an ALS catalytic subunit;
b) providing a fungal organism having a second form of the ALS catalytic subunit, wherein one of the first or the second form of the ALS catalytic subunit has at least 10% of the activity of SEQ ID NO:2; and
c) determining the pathogenicity of the organism having the first form of the ALS catalytic subunit and the organism having the second form of the ALS catalytic subunit in the presence of a test compound,
wherein a difference in pathogenicity between the two organisms in the presence of the test compound indicates that the test compound is a candidate for an antibiotic.

40. The method of claim 39, wherein the fungal organism having the first form of the ALS catalytic subunit and the fungal organism having the second form of the ALS catalytic subunit are Magnaporthe and the first and the second form of the ALS catalytic subunit are fungal ALS catalytic subunits.

41. The method of claim 39, wherein the first form of the ALS catalytic subunit is SEQ ID NO:1.

42. The method of claim 39, wherein the fungal organism having the first form of the ALS catalytic subunit and the fungal organism having the second form of the ALS catalytic subunit are Magnaporthe and the first form of the ALS catalytic subunit is SEQ ID NO:1.

43. The method of claim 39, wherein the fungal organism having the first form of the ALS catalytic subunit and the fungal organism having the second form of the ALS catalytic subunit are Magnaporthe, the first form of the ALS catalytic subunit is SEQ ID NO:1, and the second form of the ALS catalytic subunit is a heterologous ALS catalytic subunit.

44. The method of claim 39, wherein the fungal organism having the first form of the ALS catalytic subunit and the fungal organism having the second form of the ALS catalytic subunit are Magnaporthe, the first form of the ALS catalytic subunit is SEQ ID NO:1, and the second form of the ALS catalytic subunit is SEQ ID NO:1 comprising a transposon insertion that reduces or abolishes ALS catalytic subunit activity.

45. A method for identifying a test compound as a candidate for an antibiotic, comprising:
a) providing a fungal organism having a first form of an ALS regulatory subunit;
b) providing a fungal organism having a second form of the ALS regulatory subunit, wherein one of the first or the second form of the ALS regulatory subunit has at least 10% of the activity of SEQ ID NO:5; and
c) determining the growth of the organism having the first form of the ALS regulatory subunit and the organism having the second form of the ALS regulatory subunit in the presence of a test compound,
wherein a difference in growth between the two organisms in the presence of the test compound indicates that the test compound is a candidate for an antibiotic.

46. The method of claim 45, wherein the fungal organism having the first form of the ALS regulatory subunit and the fungal organism having the second form of the ALS regulatory subunit are Magnaporthe and the first and the second form of the ALS regulatory subunit are fungal ALS regulatory subunits.

47. The method of claim 45, wherein the first form of the ALS regulatory subunit is SEQ ID NO:3 or SEQ ID NO:4.

48. The method of claim 45, wherein the fungal organism having the first form of the ALS regulatory subunit and the fungal organism having the second form of the ALS regulatory subunit are Magnaporthe and the first form of the ALS regulatory subunit is SEQ ID NO:3 or SEQ ID NO:4.

49. The method of claim 45, wherein the fungal organism having the first form of the ALS regulatory subunit and the fungal organism having the second form of the ALS regulatory subunit are Magnaporthe, the first form of the ALS regulatory subunit is SEQ ID NO:3 or SEQ ID NO:4, and the second form of the ALS regulatory subunit is a heterologous ALS regulatory subunit.

50. The method of claim 45, wherein the fungal organism having the first form of the ALS regulatory subunit and the fungal organism having the second form of the ALS regulatory subunit are Magnaporthe, the first form of the ALS regulatory subunit is SEQ ID NO:3 or SEQ ID NO:4, and the second form of the ALS regulatory subunit is SEQ ID NO:3 or SEQ ID NO:4 comprising a transposon insertion that reduces or abolishes ALS regulatory subunit activity.

51. A method for identifying a test compound as a candidate for an antibiotic, comprising:
a) providing a fungal organism having a first form of an ALS regulatory subunit;
b) providing a fungal organism having a second form of the ALS regulatory subunit, wherein one of the first or the second form of the ALS regulatory subunit has at least 10% of the activity of SEQ ID NO:5; and
c) determining the pathogenicity of the organism having the first form of the ALS regulatory subunit and the organism having the second form of the ALS regulatory subunit in the presence of a test compound,
wherein a difference in pathogenicity between the two organisms in the presence of the test compound indicates that the test compound is a candidate for an antibiotic.

52. The method of claim 51, wherein the fungal organism having the first form of the ALS regulatory subunit and the fungal organism having the second form of the ALS regulatory subunit are Magnaporthe and the first and the second form of the ALS regulatory subunit are fungal ALS regulatory subunits.

53. The method of claim 51, wherein the first form of the ALS regulatory subunit is SEQ ID NO:3 or SEQ ID NO:4.

54. The method of claim 51, wherein the fungal organism having the first form of the ALS regulatory subunit and the fungal organism having the second form of the ALS regulatory subunit are Magnaporthe and the first form of the ALS regulatory subunit is SEQ ID NO:3 or SEQ ID NO:4.

55. The method of claim 51, wherein the fungal organism having the first form of the ALS regulatory subunit and the fungal organism having the second form of the ALS regulatory subunit are Magnaporthe, the first form of the ALS regulatory subunit is SEQ ID NO:3 or SEQ ID NO:4, and the second form of the ALS regulatory subunit is a heterologous ALS regulatory subunit.

56. The method of claim 51, wherein the fungal organism having the first form of the ALS regulatory subunit and the fungal organism having the second form of the ALS regulatory subunit are Magnaporthe, the first form of the ALS regulatory subunit is SEQ ID NO:3 or SEQ ID NO:4, and the second form of the ALS regulatory subunit is SEQ ID NO:3 or SEQ ID NO:4 comprising a transposon insertion that reduces or abolishes ALS regulatory subunit activity.

57. A method for identifying a test compound as a candidate for an antibiotic, comprising:
a) providing a fungal organism having a first form of a gene in the branched chain amino acid biosynthetic pathway;
b) providing a fungal organism having a second form of said gene in the branched chain amino acid biosynthetic pathway, wherein one of the first or the second form of the gene has at least 10% of the activity of a corresponding Magnaportha grisea gene; and
c) determining the growth of the organism having the first form of the gene and the organism having the second form of the gene in the presence of a test compound,
wherein a difference in growth between the two organisms in the presence of the test compound indicates that the test compound is a candidate for an antibiotic.

58. The method of claim 57, wherein the fungal organism having the first form of the gene and the fungal organism having the second form of the gene are Magnaporthe.

59. The method of claim 57, wherein the fungal organism having the first form of the gene and the fungal organism having the second form of the gene are Magnaporthe, the first form of the gene in the branched chain amino acid biosynthetic pathway is Magnaporthe grisea ketol-acid reductoisomerase, and the second form of the gene is a heterologous ketol-acid reductoisomerase.

60. The method of claim 57, wherein the fungal organism having the first form of the gene and the fungal organism having the second form of the gene are Magnaporthe, the first form of the gene in the branched chain amino acid biosynthetic pathway is Magnaporthe grisea ketol-acid reductoisomerase, and the second form of the gene is Magnaporthe grisea ketol-acid reductoisomerase comprising a transposon insertion that reduces or abolishes ketol-acid reductoisomerase protein activity.

61. The method of claim 57, wherein the fungal organism having the first form of the gene and the fungal organism having the second form of the gene are Magnaporthe, the first form of the gene in the branched chain amino acid biosynthetic pathway is Magnaporthe grisea dihydroxy-acid dehydratase, and the second form of the gene is a heterologous dihydroxy-acid dehydratase.

62. The method of claim 57, wherein the fungal organism having the first form of the gene and the fungal organism having the second form of the gene are Magnaporthe, the first form of the gene in the branched chain amino acid biosynthetic pathway is Magnaporthe grisea dihydroxy-acid dehydratase, and the second form of the gene is Magnaporthe grisea dihydroxy-acid dehydratase comprising a transposon insertion that reduces or abolishes dihydroxy-acid dehydratase protein activity.

63. A method for identifying a test compound as a candidate for an antibiotic, comprising:
a) providing a fungal organism having a first form of a gene in the branched chain amino acid biosynthetic pathway;
b) providing a fungal organism having a second form of said gene in the branched chain amino acid biosynthetic pathway, wherein one of the first or the second form of the gene has at least 10% of the activity of a corresponding Magnaportha grisea gene; and
c) determining the pathogenicity of the organism having the first form of the gene and the organism having the second form of the gene in the presence of a test compound,
wherein a difference in pathogenicity between the organism and the comparison organism in the presence of the test compound indicates that the test compound is a candidate for an antibiotic.

64. The method of claim 63, wherein the fungal organism having the first form of the gene and the fungal organism having the second form of the gene are Magnaporthe.

65. The method of claim 63, wherein the fungal organism having the first form of the gene and the fungal organism having the second form of the gene are Magnaporthe, the first form of the gene in the branched chain amino acid biosynthetic pathway is Magnaporthe grisea ketol-acid reductoisomerase, and the second form of the gene is a heterologous ketol-acid reductoisomerase.

66. The method of claim 63, wherein the fungal organism having the first form of the gene and the fungal organism having the second form of the gene are Magnaporthe, the first form of the gene in the branched chain amino acid biosynthetic pathway is Magnaporthe grisea ketol-acid reductoisomerase, and the second form of the gene is Magnaporthe grisea ketol-acid reductoisomerase comprising a transposon insertion that reduces or abolishes ketol-acid reductoisomerase protein activity.

67. The method of claim 63, wherein the fungal organism having the first form of the gene and the fungal organism having the second form of the gene are Magnaporthe, the first form of the gene in the branched chain amino acid biosynthetic pathway is Magnaporthe grisea dihydroxy-acid dehydratase, and the second form of the gene is a heterologous dihydroxy-acid dehydratase.

68. The method of claim 63, wherein the fungal organism having the first form of the gene and the fungal organism having the second form of the gene are Magnaporthe, the first form of a gene in the branched chain amino acid biosynthetic pathway is Magnaporthe grisea dihydroxy-acid dehydratase, and the second form of the gene is Magnaporthe grisea dihydroxy-acid dehydratase comprising a transposon insertion that reduces or abolishes dihydroxy-acid dehydratase protein activity.

69. A method for identifying a test compound as a candidate for an antibiotic, comprising:
a) providing paired growth media containing a test compound, wherein the paired growth media comprise a first medium and a second medium and the second medium contains a higher level of L-branched chain amino acids than the first medium;
b) innoculating the first and the second medium with an organism; and
c) determining the growth of the organism, wherein a difference in growth of the organism between the first and second medium indicates that the test compound is a candidate for an antibiotic.

70. The method of claim 69, wherein the organism is a fungus.

71. The method of claim 70, wherein the organism is Magnaporthe.

72. An isolated nucleic acid comprising a nucleotide sequence that encodes the polypeptide of SEQ ID NO:2.

73. An isolated nucleic acid comprising a nucleotide sequence that encodes the polypeptide of SEQ ID NO:5.

74. An isolated nucleic acid comprising a nucleotide sequence encoding a polypeptide having at least 50% sequence identity to SEQ ID NO:2 and having at least 10% of the activity of SEQ ID NO:2.

75. An isolated nucleic acid comprising a nucleotide sequence encoding a polypeptide having at least 50% sequence identity to SEQ ID NO:5 and having at least 10% of the activity of SEQ ID NO:5.

76. An isolated nucleic acid comprising a nucleotide sequence that encodes a polypeptide consisting essentially of the amino acid sequence of SEQ ID NO:2.

77. An isolated nucleic acid comprising a nucleotide sequence that encodes a polypeptide consisting essentially of the amino acid sequence of SEQ ID NO:5.

78. An isolated polypeptide comprising the amino acid sequence of SEQ ID NO:2.

79. An isolated polypeptide comprising the amino acid sequence of SEQ ID NO:5.

80. An isolated polypeptide consisting essentially of the amino acid sequence of SEQ ID NO:2.

81. An isolated polypeptide consisting essentially of the amino acid sequence of SEQ ID NO:5.
